(19) United States
(12) Patent Application Publication
(10) Pub. No.: US 2025/0082244 A1
(43) Pub. Date: Mar. 13, 2025

(54) WEARABLE APTAMER FIELD-EFFECT TRANSISTOR SENSING SYSTEM FOR NONINVASIVE CORTISOL MONITORING AND WEARABLE SYSTEM FOR STRESS SENSING

(71) Applicant: The Regents of the University of California, Oakland, CA (US)

(72) Inventors: Sam EMAMINEJAD, Los Angeles, CA (US); Anne ANDREWS, Los Angeles, CA (US); Bo WANG, Los Angeles, CA (US); Chuanzhen ZHAO, Los Angeles, CA (US)

(73) Assignee: The Regents of the University of California, Oakland, CA (US)

(21) Appl. No.: 18/726,685

(22) PCT Filed: Jan. 4, 2023
(86) PCT No.: PCT/US2023/010142

§ 371 Date: Jul. 3, 2024

Related U.S. Application Data
(60) Provisional application No. 63/296,447, filed on Jan. 4, 2022.

(51) Int. Cl. A61B 5/302; A61B 5/00; A61B 5/145; C12N 15/115

(52) U.S. Cl. CPC A61B 5/302; A61B 5/14507; A61B 5/14546; A61B 5/681; C12N 15/115; C12N 2310/16; C12N 2310/531

(57) ABSTRACT

Wearable technologies for personalized monitoring require sensors that track biomarkers often present at low levels. Cortisol—a key stress biomarker—is present in sweat at low nanomolar concentrations. Previous wearable sensing systems are limited to analytes in the micromolar-millimolar ranges. To overcome these and other limitations, the present embodiments include a flexible field-effect transistor (FET) biosensor array that exploits a new cortisol aptamer coupled to nanometer-thin-film In₂O₃ FETs. Cortisol levels were determined via molecular recognition by aptamers where binding was transduced to electrical signals on FETs. The physiological relevance of cortisol as a stress biomarker was demonstrated by tracking salivary cortisol levels in participants in a Trier Social Stress Test and establishing correlations between cortisol in diurnal saliva and sweat samples. These correlations motivated the development and on-body validation of an aptamer-FET array-based smartwatch equipped with a custom, multi-channel, self-referencing autonomous source measurement unit enabling seamless, real-time cortisol sweat sensing.

### CROSS-REFERENCE TO RELATED APPLICATIONS

The present application is based on and claims priority to U.S. Provisional Patent Application No. 63/296,447 filed Jan. 4, 2022, the contents of which are incorporated by reference herein in their entirety.

### STATEMENT OF GOVERNMENT SPONSORED RESEARCH

This invention was made with government support under Grant Number 1847729, awarded by the National Science Foundation and under Grant Number DA045550, awarded by the National Institutes of Health. The government has certain rights in the invention.

### TECHNICAL FIELD

The present embodiments relate generally to noninvasive biological monitoring and sensing and more particularly to a field-effect transistor (FET) array-based sensing system that exploits a cortisol aptamer coupled to nanometer-thin In₂O₃ channels of FETs.

### BACKGROUND

Wearable monitoring technologies have the power to transform healthcare by providing personalized, actionable feedback enabling changes in physical and cognitive performance and the adoption of healthier lifestyle routines. Wearable sensors that detect and quantify biomarkers in retrievable biofluids (e.g., sweat, interstitial fluid, tears, urine, blood) to provide specific information on human dynamic physiological and psychological status remain an elusive goal of such technologies. On-body sensing systems have been used to make measurements of physiologically informative indices in sweat, including pH, and electrolyte, metabolite, or nutrient levels. However, many obstacles remain to extrapolate such techniques into realizable and useful formats. Current wearable sensing systems, particularly those used for continuous monitoring, are limited to biomarkers that exist in biofluids in the micromolar-millimolar ranges. Nonetheless, many important biomarkers, including those that provide feedback on stress, inflammation, metabolic status, reproductive status, and other physiological and psychological states are present in biofluids at submicromolar concentrations. These low-concentration biomarkers are not accessible for monitoring using existing technologies.

It is against this technological backdrop that the present Applicant sought a technological solution to these and other problems rooted in this technology.

### SUMMARY

The present embodiments relate generally to monitoring low-concentration, small-molecule biomarkers, such as cortisol, in a wearable format. These and other embodiments include a FET array-based sensing system. This example array exploits a newly identified cortisol aptamer (as a biorecognition element) coupled to the nanometer-thin In₂O₃ channels of FETs (as a signal transduction platform). These and other aptamer-based FET sensors show robust and selective target detection in minimally or undiluted biological fluids, including blood, serum, plasma, urine, interstitial fluid, and sweat, and in brain tissue.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIGS. 1A to 1F illustrate example aspects of a fully autonomous non-invasive cortisol biomarker monitoring system using a wearable aptamer-field-effect transistor (FET) sensing system according to embodiments.

FIGS. 2A to 2K illustrate example aspects of flexible polyimide thin-film In₂O₃ field-effect transistors (FETs) according to embodiments.

FIGS. 3A to 3J illustrate example aspects of biological applicability of aptamer-FET sensors according to embodiments.

FIGS. 4A to 4F illustrate aspects of an example integrated aptamer-FET sensing system with an on-board source measurement unit (SMU) according to embodiments.

FIGS. 5A to 5D illustrate aspects of an example wireless and wearable aptamer-FET sensing system for on-body sweat analysis according to embodiments.

FIG. 6 is a schematic illustration of an example flexible sensor array fabrication according to embodiments.

FIGS. 7A and 7B illustrate an example atomic force microscopy characterization of In₂O₃ thin films according to embodiments.

FIGS. 8A to 8H are graphs illustrating examples of calibrated responses for FET-based pH sensors at different values of source-gate voltages (VGS) according to embodiments.

FIGS. 9A and 9B illustrate example aspects of the stability of flexible In₂O₃ FETs after bending according to embodiments.

FIGS. 10A to 10F illustrate example aspects of cortisol aptamer characterization according to embodiments.

FIG. 11 illustrates an example schematic of aptamer-FET surface functionalization according to embodiments.

FIG. 12 illustrates examples of cortisol-aptamer-FET calibrated responses in artificial saliva according to embodiments.

FIGS. 13A to 13C illustrate example aspects of the identification and characterization of serotonin in human saliva and sweat samples by LC-MS/MS according to embodiments.

FIG. 14 illustrates examples of serotonin-aptamer-FET calibrated responses in artificial sweat according to embodiments.

FIGS. 15A and 15B illustrate example correlations between salivary and sweat cortisol levels determined by enzyme-linked immunosorbent assay (ELISA) vs. field-effect transistor (FET) measurements according to embodiments.

FIG. 16 illustrates example temperature sensor responses according to embodiments.

FIGS. 17A and 17B illustrate example schematics of field-effect transistor (FET) signal acquisition (standard laboratory instrumentation) according to embodiments.

FIG. 18 illustrates an example comparison of field-effect transistor source-drain current (IDS) responses to pH changes from a custom-developed printed circuit board (PCB) vs. a multi-channel potentiostat according to embodiments.

FIGS. 19A and 19B illustrate example ex-situ effects of vortical vibration on field-effect transistor sensing responses for different microfluidic channel heights according to embodiments.

FIGS. 20A to 20L illustrate example aspects of multi-channel pH data acquisition via a flexible printed circuit board according to embodiments.

FIG. 21 illustrates example characterization of leakage current through a gate electrode according to embodiments.

FIGS. 22A to 22C illustrate example characterizations of cortisol in human saliva and sweat samples by LC-MS/MS according to embodiments.

FIGS. 23A to 23F illustrate an example summary of chemical structures according to embodiments.

FIGS. 24A and 24B provide example images for the aptamer-FET biosensing smartwatch and smartphone application according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of the exchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, Applicants do not intend for any term in the specification or claims to be ascribed an uncommon or special meaning unless explicitly set forth as such. Further, the present embodiments encompass present and future known equivalents to the known components referred to herein by way of illustration.

### Introduction

As set forth above, wearable monitoring technologies have the power to transform healthcare by providing personalized, actionable feedback enabling changes in physical and cognitive performance and the adoption of healthier lifestyle routines. Several investigations have been performed in connection with wearable sensors that detect and quantify biomarkers in retrievable biofluids so as to provide specific information on human dynamic physiological and psychological status (J. Heikenfeld, A. Jajack, B. Feldman, S. W. Granger, S. Gaitonde, G. Begtrup, B. A. Katchman, Accessing analytes in biofluids for peripheral biochemical monitoring. Nat. Biotechnol. 37, 407-419 (2019); T. R. Ray, J. Choi, A. J. Bandodkar, S. Krishnan, P. Gutruf, L. Tian, R. Ghaffari, J. A. Rogers, Bio-integrated wearable systems: A comprehensive review. Chem. Rev. 119, 5461-5533 (2019)). On-body sensing systems have been proposed to make measurements of physiologically informative indices in sweat, including pH, and electrolyte, metabolite, or nutrient levels (X. Cheng, B. Wang, Y. Zhao, H. Hojaiji, S. Lin, R. Shih, H. Lin, S. Tamayosa, B. Ham, P. Stout, K. Salahi, Z. Wang, C. Zhao, J. Tan, S. Emaminejad, A mediator-free electroenzymatic sensing methodology to mitigate ionic and electroactive interferents' effects for reliable wearable metabolite and nutrient monitoring. Adv. Funct. Mater. 30, 1908507 (2020); W. Gao, S. Emaminejad, H. Y. Y. Nyein, S. Challa, K. Chen, A. Peck, H. M. Fahad, H. Ota, H. Shiraki, D. Kiriya, Fully integrated wearable sensor arrays for multiplexed in situ perspiration analysis. Nature 529, 509-514 (2016); J. Kim, A. S. Campbell, B. E.-F. de Avila, J. Wang, Wearable biosensors for healthcare monitoring. Nat. Biotechnol. 37, 389-406 (2019); Y. Zhao, B. Wang, H. Hojaiji, Z. Wang, S. Lin, C. Yeung, H. Lin, P. Nguyen, K. Chiu, K. Salahi, X. Cheng, J. Tan, B. A. Cerrillos, S. Emaminejad, A wearable freestanding electrochemical sensing system. Sci. Adv. 6, eaaz0007 (2020)).

Nevertheless, the present Applicant recognizes that many low-concentration, potentially informative biomarkers are not accessible by wearable sensing systems. Included are hormones and other biomarkers, some of which are present at (sub)nanomolar levels in the presence of high-concentration interferants in native biofluids. Shortcomings are inherent at the sensor and systems levels. As such, the potential utility of wearable sensors remains limited to a small number of narrow applications. Moreover, existing wearable systems have neither the resolution nor dynamic capabilities needed to capture physiologically relevant changes in biomarker levels in individuals continuously, accurately, and seamlessly.

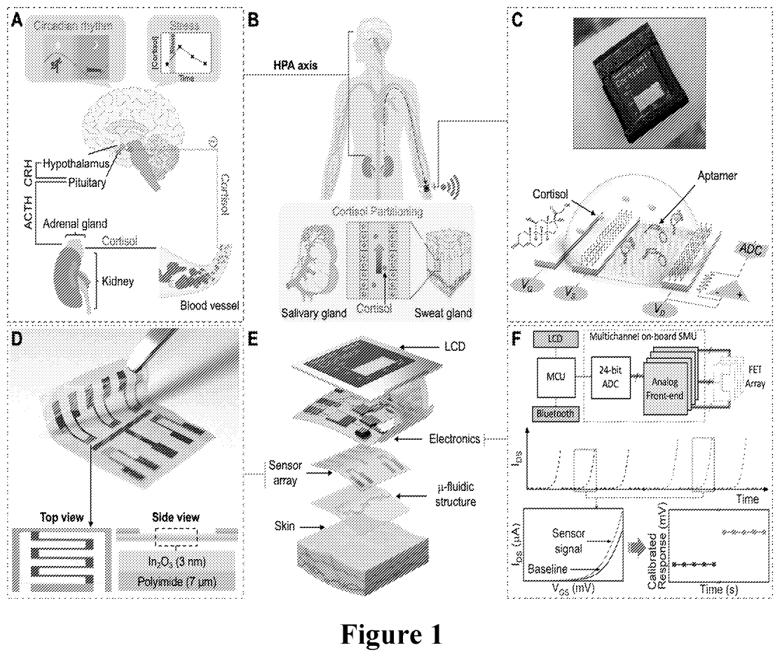

Cortisol,] is an example of a low-concentration biomarker that provides information on psychobiological states that is currently challenging for noninvasive monitoring. It is a key component of the stress-responsive hypothalamus-pituitary-adrenal axis (FIGS. 1A, 1B) (A. Clow, F. Hucklebridge, T. Stalder, P. Evans, L. Thorn, The cortisol awakening response: More than a measure of HPA axis function. Neurosci. Biobehav. Rev. 35, 97-103 (2010)). Cortisol dysregulation occurs in major depressive disorder, anxiety disorders, posttraumatic stress disorder, obesity, and Cushing's and Addison's diseases (E. K. Adam, S. Vrshek-Schallhorn, A. D. Kendall, S. Mineka, R. E. Zinbarg, M. G. Craske, Prospective associations between the cortisol awakening response and first onsets of anxiety disorders over a six-year follow-up-2013 Curt Richter Award Winner. Psychoneuroendocrinology 44, 47-59 (2014); P. Restituto, J. Galofre, M. Gil, C. Mugueta, S. Santos, J. Monreal, N. Varo, Advantage of salivary cortisol measurements in the diagnosis of glucocorticoid related disorders. Clin. Biochem. 41, 688-692 (2008); R. Yehuda, M. H. Teicher, R. L. Trestman, R. A. Levengood, L. J. Siever, Cortisol regulation in posttraumatic stress disorder and major depression: A chronobiological analysis. Biol. Psychiatry 40, 79-88 (1996); A. C. Incollingo Rodriguez, E. S. Epel, M. L. White, E. C. Standen, J. R. Seckl, A. J. Tomiyama, Hypothalamic-pituitary-adrenal axis dysregulation and cortisol activity in obesity: A systematic review. Psychoneuroendocrinology 62, 301-318 (2015)). Landmark studies have linked individual cortisol levels to neurobehavioral developmental trajectories, and personal and team performance outcomes (M. Akinola, E. Page-Gould, P. H. Mehta, J. G. Lu, Collective hormonal profiles predict group performance. Proc. Natl. Acad. Sci. USA 113, 9774-9779 (2016); S. Hart, L. M. Boylan, B. Border, S. R. Carroll, D. McGunegle, R. M. Lampe, Breast milk levels of cortisol and Secretory Immunoglobulin A (SIgA) differ with maternal mood and infant neuro-behavioral functioning. Infant Behav. Dev. 27, 101-106 (2004)). Clinical studies have demonstrated significant correlations between free cortisol levels in saliva and blood (U. Teruhisa, H. Ryoji, I. Taisuke, S. Tatsuya, M. Fumihiro, S. Tatsuo, Use of saliva for monitoring unbound free cortisol levels in serum. Clin. Chim. Acta 110, 245-253 (1981); R. F. Vining, R. A. McGinley, J. J. Maksvytis, K. Y. Ho, Salivary cortisol: A better measure of adrenal cortical function than serum cortisol. Ann. Clin. Biochem. 20, 329-335 (1983)). These associations are attributed to the relatively small size of cortisol (molecular weight 362.5 g/mol) and its lipophilicity, which enable diffusion through glandular and capillary epithelial cell membranes. Similar correlations are hypothesized for cortisol in sweat due to comparable diffusive transport mechanisms from blood to sweat (FIG. 1B) (J. Heikenfeld, Non-invasive analyte access and sensing through eccrine sweat: Challenges and outlook circa 2016. Electroanalysis 28, 1242-1249 (2016)).

Recent advances in biosensor development illustrate the importance and promise of noninvasive cortisol monitoring (O. Parlak, S. T. Keene, A. Marais, V. F. Curto, A. Salleo, Molecularly selective nanoporous membrane-based wearable organic electrochemical device for noninvasive cortisol sensing. Sci. Adv. 4, eaar2904 (2018); S. Kim, B. Lee, J. T. Reeder, S. H. Seo, S.-U. Lee, A. Hourlier-Fargette, J. Shin, Y. Sekine, H. Jeong, Y. S. Oh, Soft, skin-interfaced microfluidic systems with integrated immunoassays, fluorometric sensors, and impedance measurement capabilities. Proc. Natl. Acad. Sci. USA 117, 27906-27915 (2020); R. M. Torrente-Rodriguez, J. Tu, Y. Yang, J. Min, M. Wang, Y. Song, Y. Yu, C. Xu, C. Ye, W. W. IsHak, Investigation of cortisol dynamics in human sweat using a graphene-based wireless mHealth system. Matter 2, 921-937 (2020); A. Ganguly, K. C. Lin, S. Muthukumar, S. Prasad, Autonomous, real-time monitoring electrochemical aptasensor for circadian tracking of cortisol hormone in sub-microliter volumes of passively eluted human sweat. ACS Sens. 6, 63-72 (2021); W. Tang, L. Yin, J. R. Sempionatto, J. M. Moon, H. Teymourian, J. Wang, Touch-based stressless cortisol sensing. Adv. Mater. 33, 2008465 (2021)). Nonetheless, a wearable device for cortisol sensing employing label-free and direct signal transduction, high sensitivity and selectivity, and real sample analysis capabilities (i.e., integration with electronics such that the sensor readout is processed autonomously and communicated wirelessly) has not yet been demonstrated (see Table S1 for a comparative analysis of results from recent publications). For example, antibody-based cortisol sensors typically require the addition of external reagents and multi-step manual operations constraining applications to ex-situ settings (H.-B. Lee, M. Meeseepong, T. Q. Trung, B.-Y. Kim, N.-E. Lee, A wearable lab-on-a-patch platform with stretchable nanostructured biosensor for non-invasive immunodetection of biomarker in sweat. Biosens. Bioelectron. 156, 112133 (2020); C. Cheng, X. Li, G. Xu, Y. Lu, S. S. Low, G. Liu, L. Zhu, C. Li, Q. Liu, Battery-free, wireless, and flexible electrochemical patch for in situ analysis of sweat cortisol via near field communication. Biosens. Bioelectron. 172, 112782 (2021); P. Rice, S. Upasham, B. Jagannath, R. Manuel, M. Pali, S. Prasad, CortiWatch: Watch-based cortisol tracker. Future Sci. OA 5, FSO416 (2019)), while molecularly imprinted polymer (MIP)-based sensors can require the addition of redox probes for signal enhancement.

TABLE S1
Comparison of recent noninvasive cortisol sensing platforms.

| | | | | With an | | Detection | |
|---|---|---|---|---|---|---|---|
| | | Signal | Direct and | integrated | Human | limit or | |
| | Biorecognition | transduction | label-free | wireless | subjects | lowest conc. | |
| Reference | element | method | detection? | System? | studied? | investigated | |
| This Work | Aptamer | Field-effect | Yes | Yes | Yes | 0.001 | nM |
| | (previously | transistor (FET) | | | 88 subjects | | |
| | unreported) | | | | | | |
| Parlak et al. | Molecularly | Organic | Yes | No | Yes | 0.1 | nM |
| | imprinted | electrochemical | | | 2 subjects | | |
| | polymer (MIP) | transistor | | | | | |
| | | (OECT) | | | | | |
| Kim et al. | Antibody | Electrochemical | No | Yes | Yes | 0.221 nM | |
| | | (amperometry) | Multi-step | | 12 subjects | (0.08 ng/mL) | |
| | | | operations and | | | | |
| | | | external | | | | |
| | | | reagents | | | | |
| | | | needed. | | | | |
| Torrente- | Antibody | Colorimetric | No | Not | Yes | 13.79 nM | |
| Rodriguez et al. | | (Lateral flow | Multi-step | applicable | 4 subjects | (5 ng/mL) | |
| | | immunoassay) | operations and | | | | |
| | | | external | | | | |
| | | | reagents | | | | |
| | | | needed. | | | | |
| Ganguly et al. | Aptamer | Single-frequency | No | No | Yes | 11.03 nM | |
| | | electrochemical | | | 3 subjects | (4 ng/mL) | |
| | | impedence | | | | | |
| | | spectroscopy | | | | | |
| | | (EIS) | | | | | |
| Tang et al. | MIP | Electrochemical | No | Yes | Yes | 1 | nM |
| | | (amperometry) | Assisted by | (touch- | 7 subjects | | |
| | | | redox probes | based | | | |
| | | | (i.e., Prussian | portable | | | |
| | | | blue) | system) | | | |
| Lee et al. | Antibody | EIS | No | No | Yes | 0.0028 nM | |
| | | | External | | 3 subjects | (1 pg/mL) | |
| | | | reagent | | | | |
| | | | delivery | | | | |
| | | | needed (i.e., | | | | |
| | | | K₃[Fe(CN)₆]) | | | | |
| Cheng et al. | Antibody | Electrochemical | No | Yes | Yes | 7.47 | nM |
| | | (differential | Multi-step | | 2 subjects | | |
| | | pulse | operations and | | | | |
| | | voltammetry) | external | | | | |
| | | | reagents | | | | |
| | | | needed. | | | | |
| Rice et al. | Antibody | Electrochemical | No | Yes | Yes | 0.0028 nM | |
| | | (amperometry) | | | 1 subject | (1 pg/mL) | |
| Ishikawa et al. | Antibody | FETr | No | No | No | 0.0028 nM | |
| | | | | | | (1 pg/mL) | |
| Jang et al. | Aptamer | Electrochemical | Yes | No | Yes | 2.76 nM | |
| | | (non-faradaic | | | 5 subjects | (1 ng/mL) | |
| | | EIS) | | | | | |
| Moriarty et al. | Aptamer | Graphene FET | Yes | Yes | Yes | 0.01 | nM |
| | | | | | 1 subject | | |
| Zhang et al. | Antibody | Graphene FET | Yes | Yes | Yes | 0.028 nM | |
| | | | | | 1 subject | (10 pg/ml) | |
| Ku et al. | Aptamer | Electrochemical | No | No | No | 1 | nM |
| | | (differential | Redox probe | | | | |
| | | pulse | assisted | | | | |
| | | voltammetry) | | | | | |

According to certain aspects, to monitor low-concentration, small-molecule biomarkers, such as cortisol, in a wearable format, the present embodiments relate to a FET array-based sensing system (FIG. 1C). This array exploits a newly identified cortisol aptamer (as a biorecognition element) coupled to the nanometer-thin In₂O₃ channels of FETs (as a signal transduction platform). Aptamer-based sensors show robust and selective target detection in minimally or undiluted biological samples, including blood, serum, and brain tissue (Y. Xiao, A. A. Lubin, A. J. Heeger, K. W. Plaxco, Label-free electronic detection of thrombin in blood serum by using an aptamer-based sensor. Angew. Chem. Int. Ed. 117, 5592-5595 (2005); H. Li, P. Dauphin-Ducharme, G. Ortega, K. W. Plaxco, Calibration-Free Electrochemical Biosensors Supporting Accurate Molecular Measurements Directly in Undiluted Whole Blood. J. Am. Chem. Soc. 139, 11207-11213 (2017); K. M. Cheung, K.-A. Yang, N. Nakatsuka, C. Zhao, M. Ye, M. E. Jung, H. Yang, P. S. Weiss, M. N. Stojanović, A. M. Andrews, Phenylalanine monitoring via aptamer-field-effect transistor sensors. ACS Sens. 4, 3308-3317 (2019); N. Nakatsuka, K.-A. Yang, J. M. Abendroth, K. M. Cheung, X. Xu, H. Yang, C. Zhao, B. Zhu, Y. S. Rim, Y. Yang, P. S. Weiss, M. N. Stojanović, A. M. Andrews, Aptamer-field-effect transistors overcome Debye length limitations for small-molecule sensing. Science 362, 319-324 (2018)). The Applicants have previously reported on the use of aptamer-FETs for highly sensitive and selective detection of small-molecule targets (e.g., glucose, serotonin, dopamine, and phenylalanine) in biofluids (C. Zhao, Q. Liu, K. M. Cheung, W. Liu, Q. Yang, X. Xu, T. Man, P. S. Weiss, C. Zhou, A. M. Andrews, Narrower nanoribbon biosensors fabricated by chemical lift-off lithography show higher sensitivity. ACS Nano 15, 904-915 (2021); Q. Liu, C. Zhao, M. Chen, Y. Liu, Z. Zhao, F. Wu, Z. Li, P. S. Weiss, A. M. Andrews, C. Zhou, Flexible multiplexed In2O3 nanoribbon aptamer-field-effect transistors for biosensing. iScience 23, 101469 (2020)). Aptamer-FET detection of serotonin was stable after exposure to brain tissue (C. Zhao, K. M. Cheung, I.-W. Huang, H. Yang, N. Nakatsuka, W. Liu, Y. Cao, T. Man, P. S. Weiss, H. G. Monbouquette, A. M. Andrews, Implantable aptamer field-effect transistor neuroprobes for in vivo neurotransmitter monitoring. Sci. Adv., 10.1126/sciadv.abj7422, (2021)). Target-induced conformational rearrangements of negatively charged aptamer phosphodiester backbones produce FET surface charge perturbations, and consequently, measurable electronic signals. The aptamer-based biorecognition process relies on the formation of aptamer-target complexes, which is independent of the chemical reactivity or intrinsic charge of the target molecules.

FIGS. 1A to 1F illustrate example aspects of a non-invasive cortisol biomarker monitoring using a wearable aptamer-field-effect transistor (FET) sensing system according to embodiments. As shown in FIG. 1A, the hypothalamus-pituitary-adrenal (HPA) axis controls cortisol levels in response to circadian rhythm and stress. Other hormones in the HPA axis include adrenocorticotropic hormone (ACTH) and corticotropin-releasing hormone (CRH). As shown in FIG. 1B, the fraction of circulating cortisol not bound to blood plasma proteins is available for excretion by salivary and sweat glands. As shown in FIG. 1C, saliva and sweat samples can be analyzed by an aptamer-field-effect transistor (FET) sensing system. Top: Photograph of an aptamer-FET-enabled biosensing smartwatch. Bottom: Schematic illustration of cortisol sensing by an aptamer-FET sensor. Gate voltage (VG), source voltage (VS), drain voltage (VD), analog-digital converter (ADC). FIG. 1D is a photograph of a FET sensor array with In₂O₃ semiconductor channels fabricated on a flexible polyimide substrate. Schematic layers are not to scale. FIG. 1E is an expanded view of the key components of an aptamer-FET biosensing smartwatch including a liquid crystal display (LCD). FIG. 1F is an overview of FET-array signal acquisition via a multichannel on-board source measurement unit (SMU). Data processing is via a microcontroller unit (MCU), display, and transmission. Source-drain current (IDS), gate voltage (VGS). Photo Credit: Zhaoqing Wang, Yichao Zhao, UCLA.

These and other embodiments include fabricated aptamer-FETs on flexible polyimide substrates for wearable sensing applications (FIG. 1D) (Y. S. Rim, S.-H. Bae, H. Chen, J. L. Yang, J. Kim, A. M. Andrews, P. S. Weiss, Y. Yang, H.-R. Tseng, Printable ultrathin metal oxide semiconductor-based conformal biosensors. ACS Nano 9, 12174-12181 (2015)). Substrates were embedded in a tape-based thin-film microfluidic device to form a skin-adherable biofluid sampling, routing, and analysis module (FIG. 1E). The potential utility of using cortisol-aptamer-FET sensors to detect stress was determined by tracking salivary cortisol levels in participants in a Trier Social Stress Test (TSST), and then establishing correlations between cortisol in diurnal sweat and saliva samples.

Biologically relevant stress-associated increases in sweat cortisol levels motivated the development and on-body validation of an aptamer-FET array-based smartwatch. The wearable smartwatch was equipped with a custom on-board multi-channel source measurement unit (SMU). The SMU featured continuous, high-resolution FET transfer curve acquisition capabilities (FIG. 1F). Readouts were processed using a normalization method to mitigate device-to-device variation (F. N. Ishikawa, M. Curreli, H.-K. Chang, P.-C. Chen, R. Zhang, R. J. Cote, M. E. Thompson, C. Zhou, A calibration method for nanowire biosensors to suppress device-to-device variation. ACS Nano 3, 3969-3976 (2009)).

Among other benefits, the present approach overcomes critical shortcomings of previously reported transistor-based biosensors lacking system integration (H.-J. Jang, T. Lee, J. Song, L. Russell, H. Li, J. Dailey, P. C. Searson, H. E. Katz, Electronic cortisol detection using an antibody-embedded polymer coupled to a field-effect transistor. ACS Appl. Mater. Interfaces 10, 16233-16237 (2018); M. Pali, B. Jagannath, K.-C. Lin, S. Upasham, D. Sankhalab, S. Upashama, S. Muthukumar, S. Prasad, CATCH (Cortisol Apta WATCH): ‘Bio-mimic alarm’ to track anxiety, stress, immunity in human sweat. Electrochim. Acta 390, 138834 (2021)), which limit translation to wearable applications. By deploying a novel aptamer-FET array-based smartwatch, embodiments achieve seamless and real-time biomarker data acquisition. Aptamer-FET sensors are generalizable and modular. They can be straightforwardly adapted in wearable and mobile formats for additional physiological biomarkers, including targets at low concentrations in sweat (or other body fluids) for which there are currently no available portable measurement technologies to advance personalized precision medicine.

### Fabrication and Characterization of Flexible FETs

The present Applicants have shown that quasi-2D In₂O₃ FETs fabricated on hard and soft substrates transduce surface interactions between tethered aptamers and their targets (J. Kim, Y. S. Rim, H. Chen, H. H. Cao, N. Nakatsuka, H. L. Hinton, C. Zhao, A. M. Andrews, Y. Yang, P. S. Weiss, Fabrication of high-performance ultrathin In₂O₃ film field-effect transistors and biosensors using chemical lift-off lithography. ACS Nano 9, 4572-4582 (2015)). Large semiconductor surface-to-volume ratios enable highly efficient signal transduction between aptamer-target binding events and semiconductor electric field perturbations (e.g., charge modulation). Moreover, aptamer-FETs are sensitive to targets having little or no charge under the high ionic strength conditions typically found in body fluids.

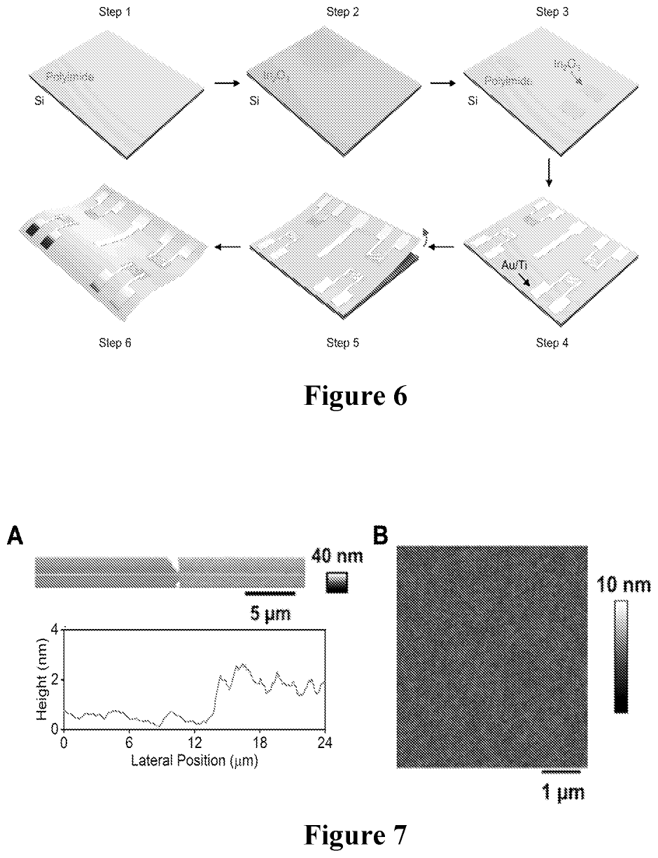

To fabricate FETs on flexible substrates for conformal skin contact, thin-film In₂O₃ was formed on polyimide via spin coating the In₂O₃ precursor followed by solution-processed sol-gel chemistry (H. Chen, Y. S. Rim, I. C. Wang, C. Li, B. Zhu, M. Sun, M. S. Goorsky, X. He, Y. Yang, Quasi-two-dimensional metal oxide semiconductors based ultrasensitive potentiometric biosensors. ACS Nano 11, 4710-4718 (2017); Y. S. Rim, H. Chen, T.-B. Song, S.-H. Bae, Y. Yang, Hexaaqua metal complexes for low-temperature formation of fully metal oxide thin-film transistors. Chem. Mater. 27, 5808-5812 (2015)). The In₂O₃ layer was then patterned by photolithography and reactive ion etching to form the channel regions (FIG. 6). Interdigitated Au/Ti electrodes were patterned to form source and drain contacts.

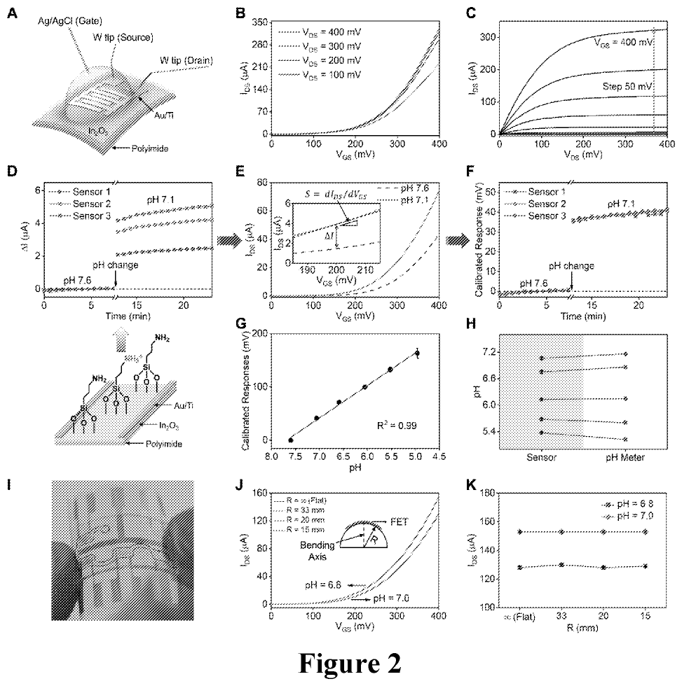

Atomic force microscopy images indicated that thin (2-3 nm) In₂O₃ films were formed on polyimide with high uniformity over relatively large areas (e.g., wafer scale) (FIG. 7). The roughness was minimal (root-mean-square roughness 0.34 nm) and comparable to the roughness of In2O3 on Si (0.4 nm). Polyimide films with FET arrays were delaminated from the underlying Si substrates for semiconductor analysis (FIG. 2A). Representative FET transfer and output characteristics are shown in FIG. 2B,C. Source-drain currents (IDS) were monitored over a range of drain voltages (VDS, 0 400 mV) and gate voltages (VGS, 0 400 mV) using a Ag/AgCl reference electrode for solution gate biasing.

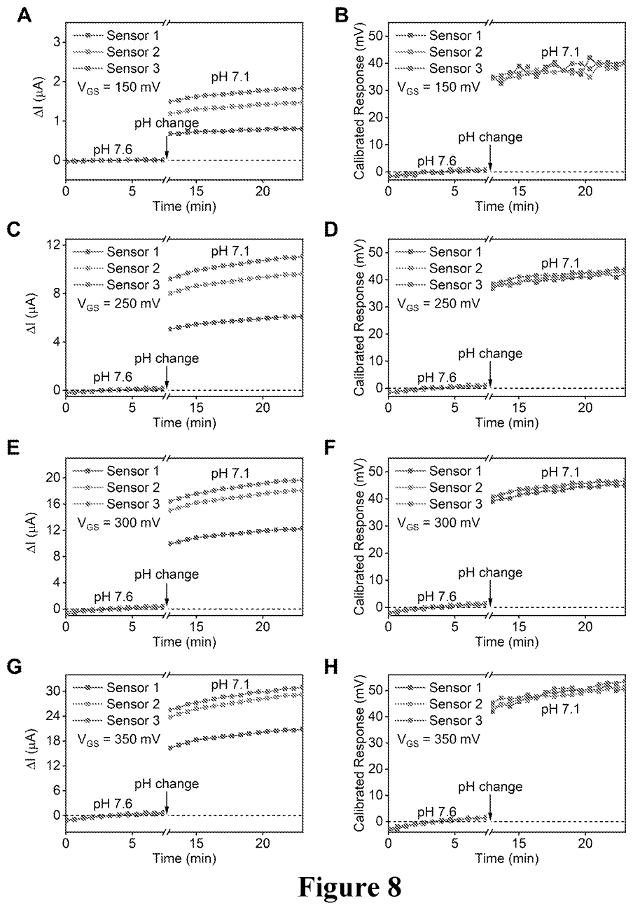

FIGS. 2A to 2K illustrate example aspects of flexible polyimide thin-film In₂O₃ field-effect transistors (FETs) according to embodiments. FIG. 2A is an example schematic of the FET setup. A Ag/AgCl reference electrode was used as the solution gate. Current between the Au/Ti source and drain electrodes was recorded via tungsten (W) probes and a semiconductor analyzer. FIG. 2B provides transfer curves (IDS VGS). The VDS was varied from 100-400 mV in 100 mV increments; the VGS was varied from 0-400 mV in 5 mV steps. FIG. 2C provides transfer curves at different VGS showing saturation behavior. The VGS was varied from 0-400 mV with 50 mV steps. FIG. 2D Top: Real-time IDS changes (ΔI) of FET-based pH sensors upon decreasing the solution pH from 7.6 to 7.1. FIG. 2D Bottom: channel surface charge perturbation mechanism. Primary amine groups of (3 aminopropyl)trimethoxysilane self-assemble on In₂O₃ and are protonated with decreasing pH (VGS=200 mV). FIG. 2E illustrates calculation of FET calibrated responses with respect to individual FET transfer characteristics. Absolute sensor responses (ΔI) were divided by the slope (S=dIDS/dVGS, a gate dependent component) to mitigate device-to-device variation. FIG. 2F provides calibrated FET pH responses (corresponding to data in FIG. 2D; VGS=200 mV). (See also, FIG. 8). FIG. 2G provides a calibration curve for FET pH sensing (N=3 FETs). FIG. 2H illustrates unknown pH values determined by FET sensors vs. a pH meter (N=3; VGS=200 mV). FIG. 2I is a photograph of a flexible FET array integrated with a tape-based microfluidic structure with the channel boundaries outlined (dotted black line). FIG. 2J provides transfer curves from a representative FET sensor at pH 6.8 or 7.0 under different bending radii. The bending axis (R) is shown in the inset. FIG. 2K illustrates the IDS output of a FET sensor (N=5 determinations for each pH condition and bending angle, VGS=400 mV). Error bars in (G), (H), and (K) are SEMs for each datum, which in some cases were too small to be displayed. VDS=400 mV for (D)-(K). Photo Credit: Zhaoqing Wang, Yichao Zhao, UCLA.

Evaluated were thin-film In₂O₃ FETs on flexible polyimide as pH sensors. The In₂O₃ was functionalized with (3-aminopropyl)triethoxysilane (APTES) diluted with trimethoxy(propyl)silane (PTMS) (1:9 v/v ratio) via self-assembly to form a pH-sensitive interface. Changes in hydrogen ion concentrations were detected via protonation/deprotonation of APTES amine tail groups (FIG. 2D), which alters surface charge to gate the underlying semiconductor. Since In₂O₃ is an n-type semiconductor, given the starting surface potential of our devices, increases in positive surface charge (i.e., increases in [H+], decreases in pH) increase IDS (P. S. Weiss, P. L. Trevor, M. J. Cardillo, Gas-surface interactions on InP monitored by changes in substrate electronic properties. J. Chem. Phys. 90, 5146-5153 (1989); A. Many, Y. Goldstein, N. B. Grover, Semiconductor Surfaces (North-Holland Publishing Co., Amsterdam, 1965)).

Decreasing the pH of the solutions above FETs over a narrow physiological range from pH 7.6 to 7.1 produced measurable increases in IDS (FIG. 2D). However, even considering differences in baseline currents at pH 7.6, pH-related changes in IDS varied across three representative FETs. Device-to-device variation is a universal drawback for FET sensors that limits their accuracy. By implementing a previously reported self-referencing method (i.e., calibrated response), we mitigated device-to-device variations (FIG. 8).

Calibrated FET responses were obtained based on the IDS VGS transfer curves by normalizing absolute changes in IDS to gate-voltage slopes at a given VGS bias (200 mV) (FIG. 2E). FIG. 2F demonstrates the use of this calibrated response method, where its application to absolute current measurements led to near identical FET calibrated responses to pH change. As shown in FIG. 8, pH-associated changes in calibrated responses calculated at different gate voltages produced similar results (VGS=150, 250, 300, or 350 mV) consistent with previous findings.

Next performed were measurements over a broader pH range from 4.6 to 7.6. The FET calibrated responses were highly linear with respect to pH (R2=0.99) with negligible device-to-device variation (FIG. 2G). The practical utility of FET pH sensors was investigated by analyzing samples with unknown pH values and cross-correlating the results with measurements obtained using a laboratory pH meter. As shown in FIG. 2H, the FET pH values closely matched the pH meter values (r=0.999, P<0.001).

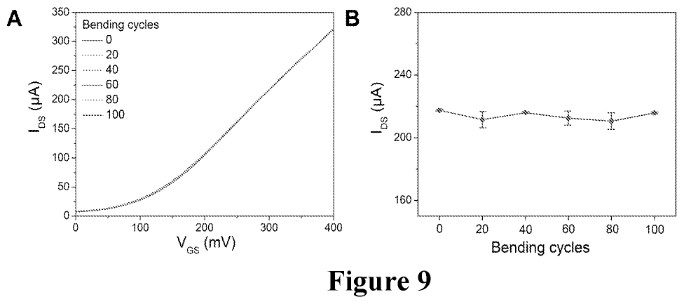

For wearable applications, investigated were the robustness of the underlying signal transduction mechanism of flexible FETs via pH sensing under mechanical deformation. Polyimide FETs were coupled to a tape-based thin-film microfluidic module (height 170 μm, FIG. 2I) to introduce pH solutions when recording sensor responses under different bending radii. Responses to pH 6.8 or pH 7.0 solutions were determined under flat and bent conditions with different curvatures (R=15, 20, or 33 mm). The FET transfer characteristics and current responses at both pH values were essentially identical regardless of the bending radii (FIG. 2J,K, respectively). Furthermore, flexible In₂O₃ FETs showed consistent transfer characteristics even after 100 bending cycles (FIG. 9) and have been previously reported to be stable after repetitive bending or crumpling with minimal mobility variations after 100 cycles.

### Development and Validation of Cortisol-Aptamer-FET Sensors

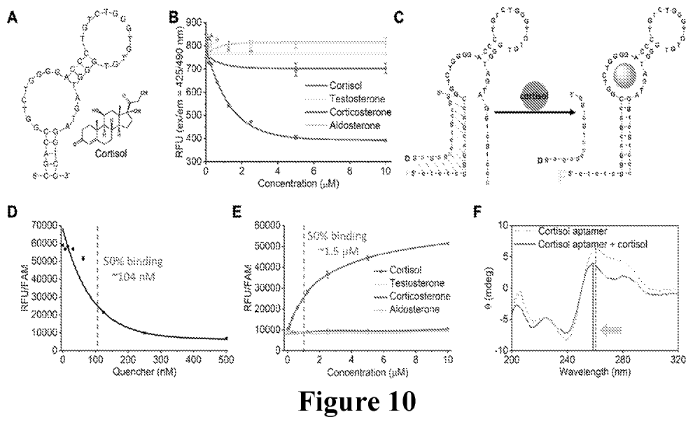

The present Applicants identified a new DNA aptamer sequence (FIG. 10A) that directly recognizes the human stress hormone cortisol using in vitro solution-phase systematic evolution of ligands by exponential enrichment (SELEX) (K.-A. Yang, H. Chun, Y. Zhang, S. Pecic, N. Nakatsuka, A. M. Andrews, T. S. Worgall, M. N. Stojanović, High-affinity nucleic-acid-based receptors for steroids. ACS Chem. Biol. 12, 3103-3112 (2017); K.-A. Yang, R. Pei, M. N. Stojanović, In vitro selection and amplification protocols for isolation of aptameric sensors for small molecules. Methods 106, 58-65 (2016)). The solution dissociation constant (Kd) of the newly identified cortisol aptamer was determined to be 500 nM via competitive fluorescence assays (FIG. 10B-10E). Demonstrated was the selectivity of the new cortisol aptamer for the target (cortisol) vs. chemically related biologically relevant non-targets (i.e., corticosterone, testosterone, and aldosterone, FIG. 10B,E). Investigated were target-induced changes in aptamer secondary structural motifs using circular dichroism spectroscopy, as in previous work (N. Nakatsuka, J. M. Abendroth, K. A. Yang, A. M. Andrews, Divalent Cation Dependence Enhances Dopamine Aptamer Biosensing. ACS Appl. Mater. Interfaces 13, 9425-9435 (2021)). Upon target association, the new cortisol aptamer showed a spectral shift and decrease in intensity in the major positive band (FIG. 10F). These spectral changes suggest a partial disruption of a parallel G quadruplex-like motif and a transition to a more extended single-stranded conformational state upon cortisol binding (J. Kypr, I. Kejnovská, D. Renčiuk, M. Vorličková, Circular dichroism and conformational polymorphism of DNA. Nucleic Acids Res. 37, 1713-1725 (2009); O. Neumann, D. Zhang, F. Tam, S. Lal, P. Wittung-Stafshede, N. J. Halas, Direct optical detection of aptamer conformational changes induced by target molecules. Anal. Chem. 81, 10002-10006 (2009)).

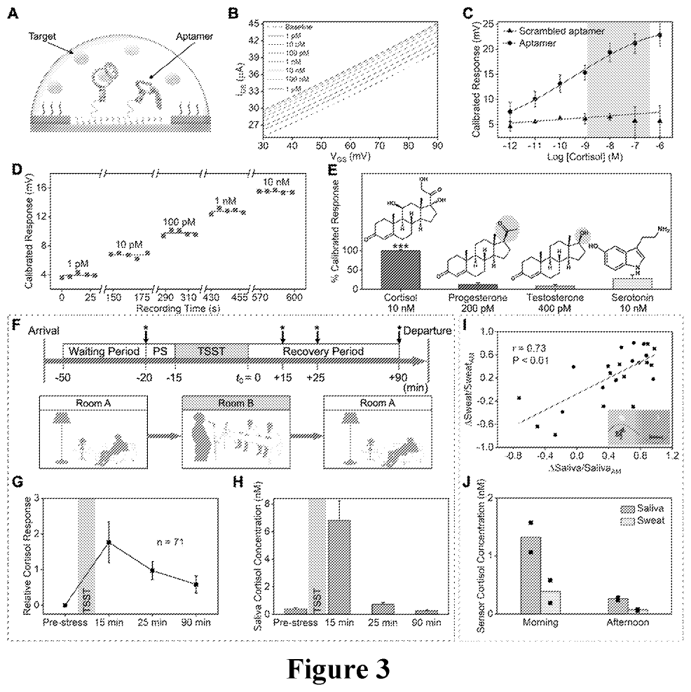

FIGS. 3A to 3J illustrate example aspects of the biological applicability of aptamer-FET sensors according to embodiments. FIG. 3A is a schematic of the aptamer-FET sensing mechanism. Cortisol-induced conformational changes occur in negatively charged aptamer phosphodiester backbones in conjunction with the rearrangement of associated solution ions. FIG. 3B provides aptamer-FET transfer curves in artificial sweat samples at varying cortisol concentrations. FIG. 3C provides Responses to cortisol for FETs functionalized with a cortisol aptamer (N=3 FETs) or a scrambled sequence (N=2 FETs) in artificial sweat. The physiologically relevant concentration range is highlighted. FIG. 3D provides time-dependent cortisol-aptamer-FET responses to artificial sweat solutions with increasing cortisol concentrations. FIG. 3E provides aptamer-FET responses to cortisol vs. non-targets in artificial sweat illustrating negligible sensor responses to the latter. ***P<0.001 vs. non-targets (N=3 FETs per target/non-target). FIG. 3F illustrates the Trier Social Stress Test protocol. The t0 is the reference timepoint corresponding to the stress period end. Starred arrows indicate saliva sampling times. Pre-stress (PS). FIG. 3G illustrates validation of the TSST protocol for eliciting cortisol responses. Cortisol was measured by standard laboratory assays. Four saliva samples were obtained at the timepoints indicated in (F) from 71 subjects. Relative cortisol responses are changes in cortisol with respect to individual pre-stress cortisol levels. FIG. 3H illustrates cortisol response of a representative TSST participant measured by cortisol-aptamer-FET sensors (N=3 replicates per timepoint; each measurement at a separate FET). FIG. 3I illustrates Morning (˜9 AM) and afternoon (˜5 PM) cortisol concentrations in sweat vs. saliva samples from 17 healthy subjects analyzed using an enzyme-linked immunosorbent assay. The ΔSweat/SweatAM and ΔSaliva/SalivaAM values were correlated and indicate decreases in cortisol levels in the afternoon with respect to the corresponding morning sample for each subject. FIG. 3J illustrates Morning and afternoon sweat/saliva cortisol levels from a representative subject measured using a cortisol-aptamer-FET. Dots represents measurements from the same sample on different devices. Error bars in (C), (E), (G), and (H) are SEMs for each datum.

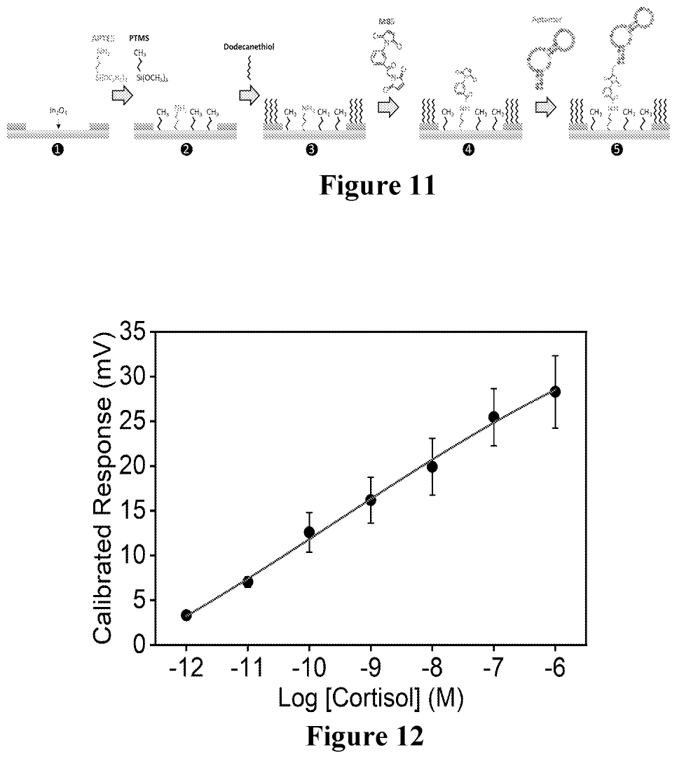

To develop an aptamer-FET sensing interface, the new cortisol aptamer with a thiol modification at the 5′ end was covalently immobilized on amino-silanized In₂O₃ FET channels using 3 maleimidobenzoic acid N hydroxysuccinimide ester (MBS) as a crosslinker (FIG. 11). Aptamer-functionalized semiconductor channels translate target binding events into measurable surface charge perturbations originating from target-induced conformational changes in the negatively charged aptamer phosphodiester backbones in conjunction with rearrangement of associated solution ions (FIG. 3A). Changes in semiconductor surface charge manifest as changes in the effective VGS, and subsequently, IDS and are quantified electronically in a label-free and reagentless manner.

FIG. 3B illustrates transfer (IDS-VGS) curves from a representative cortisol-aptamer-FET sensor in response to different cortisol concentrations in artificial sweat. Cortisol-aptamer-FETs detected cortisol concentrations over six orders of magnitude (i.e., 1 pM to 1 μM; FIG. 3C). The on-FET Kd was determined to be ˜30 pM. Similar sensing results were obtained in artificial saliva (FIG. 12). Control experiments using FETs functionalized with a scrambled cortisol aptamer sequence composed of the same numbers of each nucleotide as the correct cortisol aptamer sequence, but with a different primary sequence and predicted secondary structure, produced negligible FET responses (FIG. 3C). Time-dependent cortisol-aptamer-FET responses to increasing concentrations of cortisol are shown in FIG. 3D. These data indicate that aptamer-FETs can be used to monitor dynamic changes in cortisol concentrations.

Aptamer-FET sensor responses are inherently nonlinear due to the properties of semiconductor gating. Therefore, one cannot describe sensor sensitivity and limits of detection as for conventional devices, such as electrochemical glucose sensors (V. B. Juska, M. E. Pemble, A critical review of electrochemical glucose sensing: evolution of biosensor platforms based on advanced nanosystems. Sensors 20, 6013 (2020)). Instead, one can define the dynamic range (1 pM to 1 μM) as a critical parameter for cortisol aptamer FET biosensors, where 1 pM is the lowest practically detectable concentration. The lower detection limit of the cortisol dynamic range is similar to or lower than other reported cortisol sensing approaches. The present approach has the added benefits of being label-free and reagentless. The dynamic range covers the physiological range of cortisol in sweat and saliva (100 pM to 100 nM) (M. Trilck, J. Flitsch, D. Ludecke, R. Jung, S. Petersenn, Salivary cortisol measurement—a reliable method for the diagnosis of Cushing's syndrome. Exp. Clin. Endocrinol. Diabetes 113, 225-230 (2005); R. Miller, F. Plessow, M. Rauh, M. Gröschl, C. Kirschbaum, Comparison of salivary cortisol as measured by different immunoassays and tandem mass spectrometry. Psychoneuroendocrinology 38, 50-57 (2013); M. Jia, W. M. Chew, Y. Feinstein, P. Skeath, E. M. Sternberg, Quantification of cortisol in human eccrine sweat by liquid chromatography-tandem mass spectrometry. Analyst 141, 2053-2060 (2016)).

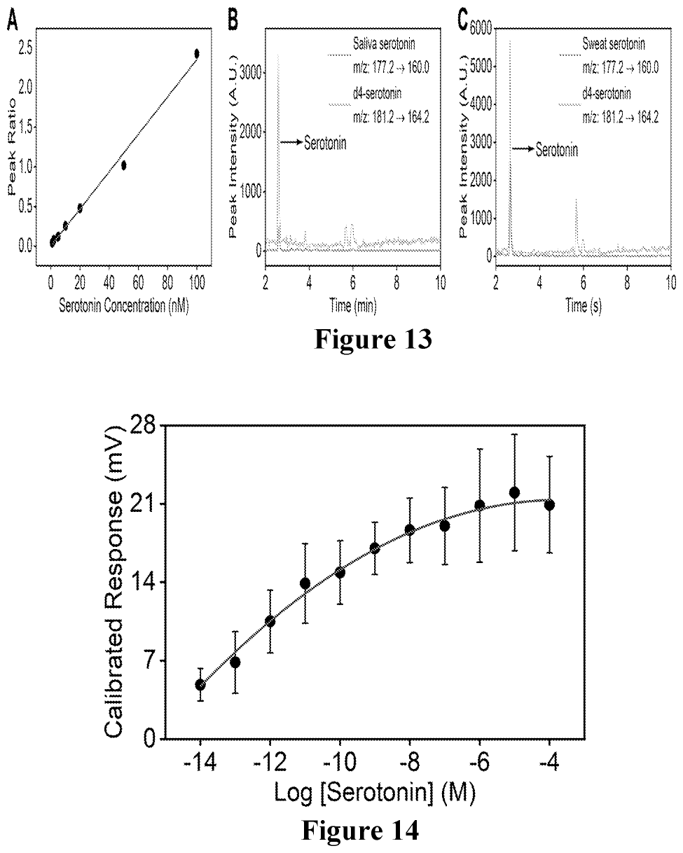

It is possible to determine the selectivity of cortisol-aptamer-FETs by measuring responses to other closely structured steroid hormones (i.e., testosterone and progesterone) and the biogenic amine serotonin, all within their physiological concentration ranges in sweat and saliva (C. Muir, K. Treasurywala, S. McAllister, J. Sutherland, L. Dukas, R. Berger, A. Khan, D. DeCatanzaro, Enzyme immunoassay of testosterone, 17β-estradiol, and progesterone in perspiration and urine of preadolescents and young adults: Exceptional levels in men's axillary perspiration. Horm. Metab. Res. 40, 819-826 (2008); K. Ngamchuea, K. Chaisiwamongkhol, C. Batchelor-McAuley, R. G. Compton, Chemical analysis in saliva and the search for salivary biomarkers—a tutorial review. Analyst 143, 81-99 (2017); Z.-L. Tan, A.-M. Bao, M. Tao, Y.-J. Liu, J.-N. Zhou, Circadian rhythm of salivary serotonin in patients with major depressive disorder. Neuroendocrinol. Lett. 28, 395-400 (2007)). Cortisol-aptamer-FETs showed negligible responses to non-targets vs. 10 nM cortisol, the estimated physiological concentrations in sweat (FIG. 3E). This aptamer-FET sensing approach can be applied, in principle, to other biomarkers in complex biological matrices by functionalizing individual FETs in arrays with different target-specific aptamers. To illustrate generalizability, measured was the target serotonin, which is also present in noninvasively retrievable biofluids such as sweat and saliva (FIG. 13), using a previously isolated serotonin aptamer. Flexible polyimide serotonin-aptamer-FETs detected serotonin in artificial sweat over a large concentration range (10 fM to 100 μM, FIG. 14), similar to the performance of serotonin-aptamer-FETs on Si or polyethylene terephthalate (PET) substrates.

Some embodiments focused on cortisol detection, as many previous studies have demonstrated the clinical significance of cortisol in a variety of contexts (e.g., as informative of stress responses and circadian rhythm). Cortisol release is mediated by the hypothalamic-pituitary-adrenal axis, which has a central role in mobilizing the body to respond to physical and psychosocial stressors (S. S. Dickerson, M. E. Kemeny, Acute stressors and cortisol responses: A theoretical integration and synthesis of laboratory research. Psychol. Bull. 130, 355-391 (2004)), as well as to disease and injury via inflammation (FIG. 1A,B) (A. Papadimitriou, K. N. Priftis, Regulation of the hypothalamic-pituitary-adrenal axis. Neuroimmunomodulation 16, 265-271 (2009)). Normal cortisol levels follow a diurnal pattern where concentrations peak shortly after waking and then decline during the day (C. A. Elverson, M. E. Wilson, Cortisol: Circadian rhythm and response to a stressor. Newborn Infant Nurs. Rev. 5, 159-169 (2005)).

Physiological and psychosocial stressors disturb circadian cortisol levels resulting in transient elevations (M. A. Birkett, The Trier Social Stress Test protocol for inducing psychological stress. J. Vis. Exp. 56, e3238 (2011)). Cortisol levels vary greatly across people, and we anticipate that the ability to monitor individual cortisol levels will provide useful information for personalized medicine (U. Knutsson, J. Dahlgren, C. Marcus, S. Rosberg, M. Bronnegird, P. Stierna, K. Albertsson-Wikland, Circadian cortisol rhythms in healthy boys and girls: Relationship with age, growth, body composition, and pubertal development. J. Clin. Endocrinol. Metab. 82, 536-540 (1997); J. M. Smyth, M. C. Ockenfels, A. A. Gorin, D. Catley, L. S. Porter, C. Kirschbaum, D. H. Hellhammer, A. A. Stone, Individual differences in the diurnal cycle of cortisol. Psychoneuroendocrinology 22, 89-105 (1997)). Information on cortisol levels can be gleaned noninvasively on a person-by-person basis by making measurements in peripheral, easily accessible biofluids, such as saliva or sweat.

Some embodiments employed the Trier Social Stress Test (TSST), a gold-standard laboratory procedure used to induce stress reliably in human participants to establish stress-induced increases in salivary cortisol. The TSST consisted of 1) test environment acclimation; 2) a pre-stress period when participants were informed about the upcoming task; 3) a stress period where participants were asked to deliver a speech and then to respond verbally to a challenging arithmetic problem in the presence of two evaluators; and 4) a recovery period (FIG. 3F). Saliva samples were collected from 71 healthy participants at four time points (i.e., pre-stress, and 15, 25, and 90 min after stress). Salivary cortisol levels were quantified by a standard laboratory assay (i.e., liquid chromatography with tandem mass spectrometry, LC-MS/MS, and enzyme-linked immunosorbent assay, ELISA).

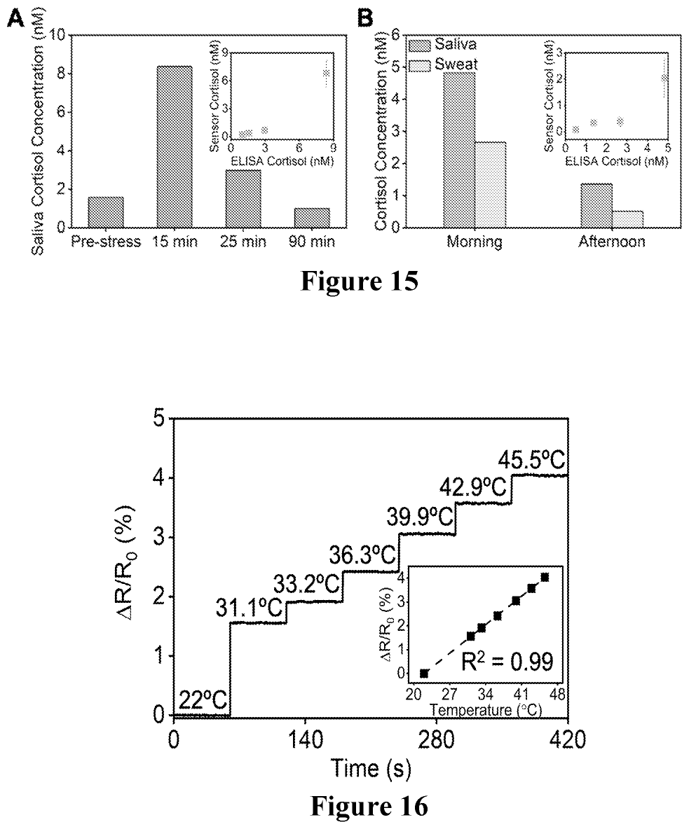

Salivary cortisol concentrations peaked 15 min after the stress period and then declined over 75 min (FIG. 3G). Analyzed were the saliva samples from a representative TSST participant using a cortisol-aptamer-FET device. The FET sensor measurements also revealed a cortisol peak 15 min after stress, followed by cortisol recovery to baseline 90 min after stress (FIG. 3H) in agreement with the aggregated trend demonstrated by the standard lab assays (FIG. 15).

For wearable applications, establishing a saliva-sweat correlation is crucial as it enables leveraging existing knowledge of salivary biomarkers (E. Kaufman, I. B. Lamster, The diagnostic applications of saliva—a review. Crit. Rev. Oral. Biol. Med. 13, 197-212 (2002); J. M. Yoshizawa, C. A. Schafer, J. J. Schafer, J. J. Farrell, B. J. Paster, D. T. Wong, Salivary biomarkers: Toward future clinical and diagnostic utilities. Clin. Microbiol. Rev. 26, 781-791 (2013)) as a foundation for future directions for sweat-based wearable applications. As such, a saliva-sweat correlation study was performed. Saliva and sweat samples were collected from 17 healthy subjects at two time points during the day (i.e., ˜9 AM and ˜5 PM). These times were selected as they are roughly the peak and nadir for diurnal variations in human cortisol levels. All samples were analyzed by ELISA. Most participants had higher saliva and sweat cortisol levels in the morning vs. afternoon, in agreement with previous saliva cortisol studies. The correlation between salivary and sweat cortisol levels was 0.73 (FIG. 3I) supporting a correlation between salivary and sweat cortisol levels.

Cortisol-aptamer-FETs were used to determine diurnal variations in cortisol levels from saliva and sweat samples from a representative subject. The FET sensor responses showed elevated (morning) and decreased (afternoon) cortisol levels reflected in saliva and sweat samples (FIG. 3J), consistent with the observations made by analyzing the same samples by ELISA (FIG. 15).

### Wireless Aptamer-FET Sensing System for Wearable Sweat Analysis

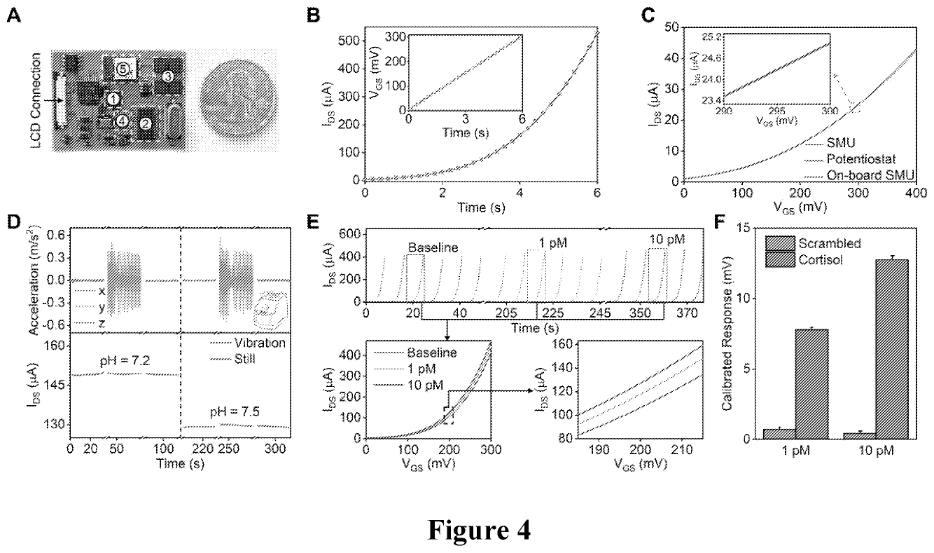

Detecting biologically relevant differences in cortisol in sweat using aptamer-FETs suggested utility for personal biomonitoring. These findings motivated the development of a wearable FET-array sensing system to track sweat cortisol and pH levels seamlessly. A FET functionalized with a scrambled cortisol aptamer sequence was included in the array to measure nonspecific responses. To illustrate versatility, also included was a temperature sensor (FIG. 16). A representative multi-channel flexible printed circuit board (FPCB) was designed to interface with the sensing array as illustrated in FIG. 4A.

More particularly, FIGS. 4A and 4B illustrate an example integrated aptamer-FET sensing system with on-board source measurement unit (SMU) according to embodiments. FIG. 4A is a Photograph of the flexible printed circuit board (FPCB) next to a U.S. quarter. The components are: 1) microcontroller unit (MCU); 2) analogue-to-digital converter (ADC); 3) potentiostat chip; 4) digital to analog converter (DAC); and 5) bluetooth. FIG. 4B is a Real-time sweep of VGS and recording of IDS to construct FET transfer curves measured by the SMU. FIG. 4C provides a Comparison of FET transfer curves determined by a commercial SMU (Keithley 4200A-SCS, Tektronix, Beaverton, OR), a multichannel potentiostat (CHI1040C, CH Instrument, Austin, TX), and the on-board SMU. FIG. 4D provides an Ex-situ characterization of the FET sensing system with and without vortical vibration (microfluidic channel height: 170 μm). The recording was paused in between conditions to save sensor readouts and to distinguish scenarios. Vibrational acceleration profiles are presented on the top and sensor responses are displayed on the bottom when tested in pH 7.2 and pH 7.5 solutions. FIG. 4E provides a representative real-time recording of IDS during VGS sweeps (top) to track dynamic variations in FET transfer curves in response to blank (baseline), 1 pM, or 10 pM cortisol solutions in artificial sweat recorded by the on-board SMU. (Bottom) Overlaid representative cortisol aptamer-FET transfer curves corresponding to the different solutions (higher resolution plots on the bottom right illustrate that the transfer curves are distinguishable). FIG. 4F provides a Comparison of cortisol aptamer-FET and scrambled oligonucleotide-FET (control) calibrated responses to 1 pM or 10 pM cortisol solutions in artificial sweat simultaneously recorded by the multi-channel on-board SMU. Photo Credit: Zhaoqing Wang, UCLA.

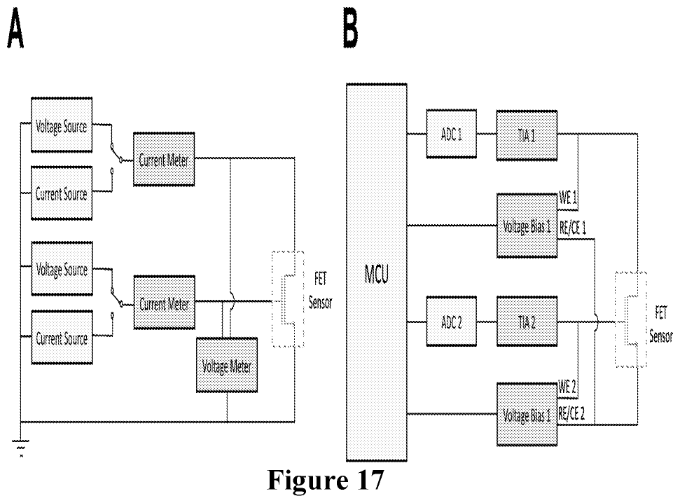

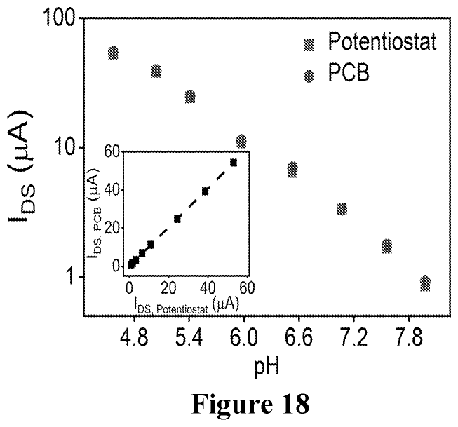

The analog front-end was dedicated to FET sensor response acquisition and was implemented as a high-resolution source measurement unit (SMU). FIG. 4B illustrates a representative on-board SMU sweep of VG (with respect to a biased VS) and recording of IDS to acquire a FET transfer curve (6 s). Tested was a commercial solid-state FET device (ADL110800) and the transfer curves obtained by the present SMU was compared with those captured by a commercial SMU (Keithley 4200A-SCS, Tektronix, Beaverton, OR) or a multichannel potentiostat (CHI1040C, CH Instruments, Austin, TX). The block diagrams of the standard laboratory instruments are shown in FIG. 17. The transfer curves measured by all three instruments were closely matched (FIG. 4C), demonstrating the FET control/signal acquisition capability of our on-board SMU. An anisotropic conductive film (ACF) was used to establish electrical connection between the FPCB and the disposable sensing array forming a sensing system for reliable signal acquisition. For validation, pH sensing using the present FPCB/SMU was compared to results obtained from a commercial multi-channel potentiostat (FIG. 18).

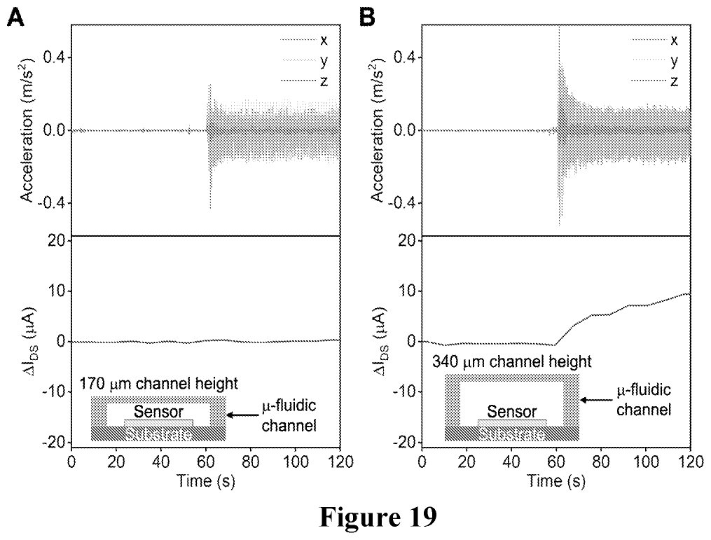

For on-wrist sweat applications involving arm movements, a tape-based thin-film microfluidic module was coupled to the FET sensing array. Evaluated was the robustness of the signal acquisition by the integrated microfluidic sensing system in the presence of motion artifacts by wirelessly recording (via bluetooth) the real-time IDS of a representative FET-based pH sensor under oscillatory motion (amplitude: ˜3 m/s2 at 5 Hz, generated by a vortex mixer) (H. Pontzer, J. H. Holloway, D. A. Raichlen, D. E. Lieberman, Control and function of arm swing in human walking and running. J. Exp. Biol. 212, 523-534 (2009)). Characterization suggested a higher degree of signal robustness for a thinner microfluidic channel (FIG. 19). Sensor responses exhibited negligible fluctuations (˜1%) despite the motion (FIG. 4D) indicating that high-fidelity measurements were achieved by the complete system, in agreement with recent studies.

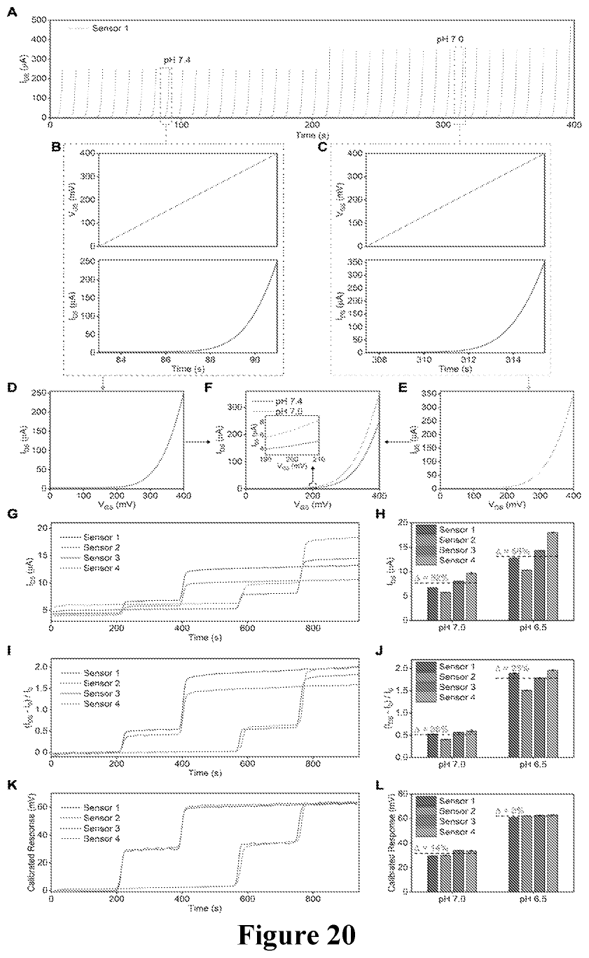

Investigated was the simultaneous multi-channel FET array response acquisition and the effectiveness of the calibrated response method to mitigate FET sensor variability using two FET-based pH sensor arrays each containing two FETs (FIG. 20) Time-dependent IDS was monitored at baseline (pH 7.4) and in response to pH decreases (pH 7.0, 6.5) at FETs in each array. Baseline normalization resulted in a reduction in device-to-device variation from ˜50% to ˜30% (FIG. 20I,J). Using calibrated responses, variability across FETs was decreased to <10% (FIG. 20K,L).

To test the capability of the sensor system to distinguish low levels of cortisol, used was a cortisol-aptamer-FET to track solution concentration changes. Real-time sweeps of VGS and recordings of IDS demonstrated that cortisol-aptamer-FETs detected cortisol as low as 1 pM (FIGS. 4E, 4F). As shown, the response time of the sensors is on the scale of seconds, while cortisol levels change in response to stress on the order of minutes to hours (FIGS. 3G, 3H). Leveraging the capability of the wearable system to measure from multiple aptamer-FETs simultaneously (i.e., from FETs functionalized with correct cortisol aptamer or scrambled cortisol aptamer sequences that function as control sensors), we found that FETs functionalized with the scrambled oligonucleotid showed comparatively negligible responses (FIG. 4F).

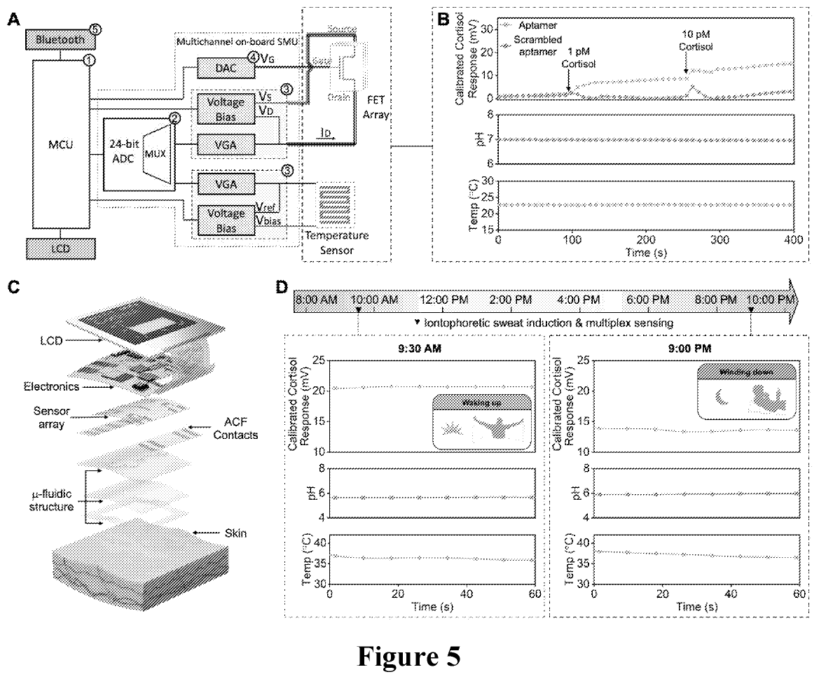

FIGS. 5A to 5D illustrate an example wireless and wearable aptamer-FET sensing system for on-body sweat analysis according to embodiments. FIG. 5A is a Systems-level block diagram of the custom-developed wireless flexible printed circuit board (FPCB), equipped with an on-board source measurement unit (SMU) for programmable, multi-channel, and high resolution 24 bit analog-digital converter biosensing. Signals acquired and processed by the FPCB were displayed by a liquid crystal display (LCD) and transmitted via bluetooth to a smartphone. FIG. 5B provides Representative real-time, multi-channel ex-situ measurements of cortisol solutions in artificial sweat, control, pH, and temperature captured by the on-board SMU. Responses at an active sensor functionalized with the correct cortisol aptamer are compared to responses at an inactive sensor functionalized with an incorrect (scrambled) sequence. FIG. 5C provides an expanded view of the wearable sensing system where the sensor array, microfluidic module, FPCB, and LCD components are integrated to form a multichannel biosensing smartwatch. FIG. 5D illustrates Real-time in-situ monitoring of natural sweat cortisol, pH, and skin temperature from a healthy subject at two time-points (9:30 AM and 9:00 PM) during routine daily activities with the multichannel biosensing smartwatch. Cortisol responses were obtained by subtracting the control channel reading (scrambled-oligonucleotide-FET) from the cortisol channel reading (cortisol-aptamer-FET).

More particularly, FIGS. 5A and 5B illustrate the integrated sensing capability for measuring cortisol (i.e., artificial sweat progressively spiked with 1 pM and 10 pM cortisol compared to a control sensor having a scrambled aptamer sequence that does not recognize cortisol), as well as simultaneous pH and temperature measurements. Incorporated was a microfluidic module and a liquid crystal display (LCD) powered by a 110 mAh lithium polymer battery to produce a “smartwatch” (FIG. 5C). With a mobile phone application, the smartwatch acquired real-time measurements (i.e., cortisol, pH, and temperature) at set time intervals. The watch was programmed to take readings in the morning (9:30 AM) and evening (9:00 PM). To access sweat, iontophoretic stimulation was performed using a Macroduct Sweat Collection System (ELITechGroup Inc., Puteaux, France) on the volar surface of the forearm of the subject. The smartwatch was then placed on the stimulated area to collect, route, and analyze the secreted sweat. FIG. 5D shows the real-time smartwatch recordings. The cortisol channel detected a decrease in the nighttime sweat cortisol level, in line with the typical circadian rhythm and observations from our ex-situ correlation study (FIGS. 3I, 3J).

### DISCUSSION

The present embodiments include a fully integrated microfluidic sensing system capable of low concentration biomarker data acquisition that enabled the direct readout of a target biomarker (cortisol) concentration in a sample-to-answer manner (via dedicated electronics) suitable for wearable applications. The present approach simultaneously overcomes several important limitations associated with recently published sweat cortisol monitoring platforms (Table S1) as it employs label-free detection, the sensing system is autonomous and wireless, the cortisol detection limits are ultra-low (1 pM), and validated was sweat cortisol as a stress biomarker in a large clinical study. Readouts from standard methods vs. aptamer-FET sensors revealed strong empirical correlations between cortisol levels in saliva and sweat samples in a pilot study. These results indicated the potential of sweat cortisol monitoring for translational applications, particularly considering an established body of knowledge related to salivary cortisol levels.

Aptamer-FETs are sensitive to environmental pH, because changes in local ion concentrations, including [H+], are detected by FETs. Thus, for translation, developed was an aptamer-FET array-based smartwatch equipped with high-resolution, multi-channel biomarker data acquisition for the simultaneous, real-time, and seamless readout of cortisol levels, pH, and temperature. The generalizability of this FET sensing system enables adaptation to a wide range of target molecules using target-specific aptamers or other receptors (e.g., antibodies) (Q. Liu, N. Aroonyadet, Y. Song, X. Wang, X. Cao, Y. Liu, S. Cong, F. Wu, M. E. Thompson, C. Zhou, Highly sensitive and quick detection of acute myocardial infarction biomarkers using In2O3 nanoribbon biosensors fabricated using shadow masks. ACS Nano 10, 10117-10125 (2016); N. Aroonyadet, X. Wang, Y. Song, H. Chen, R. J. Cote, M. E. Thompson, R. H. Datar, C. Zhou, Highly scalable, uniform, and sensitive biosensors based on top-down indium oxide nanoribbons and electronic enzyme-linked immunosorbent assay. Nano Lett. 15, 1943-1951 (2015)) that facilitate measurable surface charge perturbations in response to target-receptor interactions. The present Applicant is currently testing newly identified aptamers for additional stress biomarkers (e.g., epinephrine, norepinephrine). Once validated, these aptamers can be coupled with FETs in an array format to enable simultaneous quantification of multiple biomarkers to provide a more comprehensive view of the physiological status of users.

To enable translation of this technology into health and performance monitoring/optimization applications, dedicated and coordinated engineering and clinical efforts are required. To access target biomarker information on-demand in sedentary individuals, an iontophoresis interface will be needed to induce sweat secretion (S. Emaminejad, W. Gao, E. Wu, Z. A. Davies, H. Y. Y. Nyein, S. Challa, S. P. Ryan, H. M. Fahad, K. Chen, Z. Shahpar, Autonomous sweat extraction and analysis applied to cystic fibrosis and glucose monitoring using a fully integrated wearable platform. Proc. Natl. Acad. Sci. USA 114, 4625-4630 (2017); H. Lin, J. Tan, J. Zhu, S. Lin, Y. Zhao, W. Yu, H. Hojaiji, B. Wang, S. Yang, X. Cheng, Z. Wang, E. Tang, C. Yeung, S. Emaminejad, A programmable epidermal microfluidic valving system for wearable biofluid management and contextual biomarker analysis. Nat. Commun. 11, 4405 (2020); H. Hojaiji, Y. Zhao, M. C. Gong, M. Mallajosyula, J. Tan, H. Lin, A. M. Hojaiji, S. Lin, C. Milla, A. M. Madni, S. Emaminejad, An autonomous wearable system for diurnal sweat biomarker data acquisition. Lab Chip 20, 4582-4591 (2020)). For applications requiring continuous and prolonged biomarker sensing (e.g., athletic performance monitoring), sensor development efforts will need to focus on preserving sensor stability (e.g., anti-biofouling strategies). In-situ characterization of sweat secretion profiles (e.g., sweat rate, volume loss, etc.) will be helpful in normalizing readings for inter/intra individual physiological variations and gland activity variability.

Currently, an aptamer-FET biosensors according to embodiments is positioned for single point measurements. However, aptamer-based biosensors have been successfully regenerated (B. R. Baker, R. Y. Lai, M. S. Wood, E. H. Doctor, A. J. Heeger, K. W. Plaxco, An electronic, aptamer-based small-molecule sensor for the rapid, label-free detection of cocaine in adulterated samples and biological fluids. J. Am. Chem. Soc. 128, 3138-3139 (2006); Y. Xiao, R. Y. Lai, K. W. Plaxco, Preparation of electrode-immobilized, redox-modified oligonucleotides for electrochemical DNA and aptamer-based sensing. Nat. Protoc. 2, 2875-2880 (2007)) and utilized for continuous analyte monitoring. We have shown here and in previous work that gate voltage sweeps vs. static gate-voltage bias produce different sensor behaviors. Although the sensing mechanism of aptamer-FETs relies on surface-charge redistribution induced by target-induced changes in aptamer conformations, gate voltage also affects aptamer configurations. For example, gate voltage impacts the local electronic environment of aptamers, and when changed, (e.g., during sweeps), gate voltage can modulate aptamer conformations to low affinity states to release targets and thus, to regenerate sensors. Further investigation of the present sensing system will involve aptamer-FET measurements in larger numbers of clinical samples and continuous monitoring of cortisol fluctuations that involve decreases, as well as increases. If large mechanical deformations of the sensing platform are anticipated, further optimization will be needed to preserve the fidelity of data acquisition from both biosensor fabrication and system integration aspects.

From a clinical standpoint, given that sweat is a relatively underexplored biofluid, developing standard protocols will be advantageous (e.g., sweat-based TSST) to form the basis for large-scale, ambulatory, and longitudinal investigations centered on sweat-based biomarker studies. Accordingly, the advantages of our technology in terms of its ease of integration with wearable consumer electronics can be leveraged to facilitate such investigations (S. Lin, W. Yu, B. Wang, Y. Zhao, K. En, J. Zhu, X. Cheng, C. Zhou, H. Lin, Z. Wang, H. Hojaiji, C. Yeung, C. Milla, R. W. Davis, S. Emaminejad, Noninvasive wearable electroactive pharmaceutical monitoring for personalized therapeutics. Proc. Natl. Acad. Sci. USA 117, 19017-19025 (2020)). Large clinical datasets will enable physiological/psychobiological interpretations of sweat biomarker readings. These data can be contextualized to other user-specific static and dynamic information to render objective criteria for monitoring disease status (e.g., hormone imbalance disorders such as Cushing's disease and Addison's disease, assisting in the diagnosis of depressive disorders), as well as to provide personalized feedback to users to inform timely interventions (e.g., anxiety management via mindfulness or exercise) (P. Blanck, S. Perleth, T. Heidenreich, P. Kröger, B. Ditzen, H. Bents, J. Mander, Effects of mindfulness exercises as stand-alone intervention on symptoms of anxiety and depression: Systematic review and meta-analysis. Behav. Res. Ther. 102, 25-35 (2018)). Importantly, for wearable applications, monitoring relative changes in biomarkers in an individual over time is more important for personalized feedback than absolute determinations. For example, one commercial wearable product, Oura ring (Oura Health, Oulu, Finland), monitors nightly average body temperature variations based on a baseline determined in each user, instead of absolute temperature values. Relative temperature monitoring based on modest individual fluctuations was found to be useful for menstrual cycle tracking (A. Maijala, H. Kinnunen, H. KoskimAki, T. Jämsä, M. Kangas, Nocturnal finger skin temperature in menstrual cycle tracking: ambulatory pilot study using a wearable Oura ring. BMC Women's Health 19, 1-10 (2019)). Through convergent efforts, non-invasive monitoring modalities will be established that can be leveraged to improve the productivity and health of individuals and society.

### Example Methods

FIG. 6 is a schematic illustration of an example flexible sensor array fabrication according to embodiments. (Step 1): Polyimide was formed on silicon (Si) substrates. (Step 2): A solution of indium(III) nitrate was spin-coated and thermally processed to form a thin layer of In₂O₃. (Step 3): The In₂O₃ layer was patterned via photolithography and dry etching. (Step 4): Electron-beam metal evaporation was used to produce Au/Ti source and drain electrodes patterned via photolithography. (Step 5): Polyimide layers were delaminated from Si substrates to obtain flexible sensor arrays.

FIGS. 7A and 7B provide an example Atomic force microscopy characterization of In₂O₃ thin films according to embodiments. FIG. 7A provides Atomic force microscope images of an In₂O₃ thin-film (˜2-3 nm). The top image shows a step created by photolithography and dry etching as described in Methods, where polyimide/Si is on the left and In₂O₃/polyimide/Si is on the right. The bottom image shows the height profile along the dotted line shown in the top image. FIG. 7B illustrates Uniform deposition of In₂O₃ over a large surface area (root-mean-square roughness 0.34 nm).

FIGS. 8A to 8H are graphs illustrating Calibrated responses for field-effect transistor (FET)-based pH sensors at different gate voltage values (V_{GS}) according to embodiments. FIGS. 8A, 8C, 8E and 8G provide Real-time source-drain current (I_{DS}) changes (ΔI) for FET-based pH sensors associated with decreasing solution pH from 7.6 (baseline) to 7.1. V_{GS}=150, 250, 300, or 350 mV. FIGS. 8B, 8D, 8F and 8H provide Corresponding calibrated responses for the FET-based pH sensors (V_{GS}=150, 250, 300, or 350 mV).

FIGS. 9 A and 9B illustrate Electronic stability of flexible In₂O₃ FETs after repeated bending cycles according to embodiments. FIG. 9A provides Transfer characteristics of a representative flexible In₂O₃ transistor before bending and after bending 20, 40, 60, 80, or 100 times. The bending radius was ˜15 mm. FIG. 9B provides Corresponding source-drain currents (I_{DS}) of the flexible In₂O₃ transistor (plotted at V_{GS}=300 mV) after the different numbers of bending cycles shown in FIG. 9A. Error bars are standard errors of the means for N=3 repeated measurements.

FIGS. 10A to 10F illustrate example Cortisol aptamer characterization according to embodiments. FIG. 10A illustrates Sequence and predicted secondary structure (via mfold) for the cortisol-specific aptamer. The inset shows the chemical structure of cortisol. As shown in FIG. 10B, Using the sequence in FIG. 10A, carried out was a thioflavin T (ThT) dye displacement to compare target (cortisol) vs. non-target (testosterone, corticosterone, aldosterone) binding to the cortisol aptamer. Cortisol displaced aptamer-bound ThT producing a decrease in ThT fluorescence while non-targets did not displace ThT over the concentration range tested. FIG. 10C illustrates the aptamer sensor format for the fluorescein amidite (F) sensor/dabcyl (D) quencher structure-switching assay. Under competitive conditions, aptamer-target binding causes the aptamer to be released from the quencher strand such that the magnitude of the fluorescence response increases with increasing target concentration. FIG. 10D illustrates the association of a fluorescein (FAM)-conjugated aptamer with the (dabcyl) DAB-conjugated quencher strand was first measured in the absence of target. FIG. 10E illustrates The fluorescence response of the aptamer to cortisol and non-targets (testosterone, corticosterone, aldosterone). The K_{d} was calculated based on the plots in FIGS. 10D and 10E where: K_{d,eff1}=[free aptamer][free quencher]/[aptamer-quencher]=79 nM, K_{d,eff2}=[free quencher][aptamer-target]/[aptamer-quencher][target]=0.1525 (unitless constant), and K_{d}=K_{d,eff1}/K_{d,eff2}=79 nM/0.1525=500 nM. FIG. 10F illustrates Circular dichroism spectra of the cortisol aptamer in artificial sweat before and after incubation with cortisol.

FIG. 11 is an example Schematic of aptamer-field-effect transistor surface functionalization according to embodiments. The In₂O₃ channel (step 1) was silanized with (3-aminopropyl)triethoxysilane (APTES) and trimethoxy(propyl)silane (PTMS) (1:9) via self-assembly (step 2). The Au/Ti source and drain electrodes were passivated via self-assembly of 1-dodecanethiol monolayers (step 3). Amine-terminated silane molecules were reacted with 3-maleimidobenzoic acid N-hydroxysuccinimide ester (MBS) (step 4) to immobilize the thiolated cortisol aptamer (step 5).

FIG. 12 illustrates example Cortisol-aptamer-field-effect transistor (FET) calibrated responses in artificial saliva according to embodiments. Cortisol in artificial saliva was added to polydimethylsiloxane wells above FETs in increasing concentrations (1 pM to 1 μM). Error bars are standard errors of the means for determinations from N=3 FETs.

FIGS. 13A to 13C illustrate example Identification and characterization of serotonin in human saliva and sweat samples by liquid chromatography tandem mass spectrometry (LC-MS/MS) according to embodiments. FIG. 13A illustrates a LC-MS/MS-based serotonin calibration plot. FIG. 13B provides Ion chromatograms of serotonin in a diluted human saliva sample. FIG. 13C provides Ion chromatograms of serotonin in a diluted human sweat sample.

FIG. 14 provides example Serotonin-aptamer-field-effect transistor (FET) calibrated responses in artificial sweat according to embodiments. Serotonin in artificial sweat was added to polydimethylsiloxane wells above FETs in increasing concentrations (10 fM to 100 μM). Error bars are standard errors of the means for determinations from N=3 FETs.

FIGS. 15A and 15B illustrate Correlations between salivary or sweat cortisol levels determined by enzyme-linked immunosorbent assay (ELISA) vs. field-effect transistor (FET) measurements according to embodiments. FIG. 15A illustrates Salivary cortisol levels from a representative Trier Social Stress Test participant analyzed by ELISA. Inset: correlation between cortisol levels measured by a FET sensor (data shown in FIG. 3H) (r=0.98, P<0.05). FIG. 15B illustrates Morning and afternoon saliva and sweat cortisol levels from a representative participant analyzed by ELISA. Inset: correlation with cortisol levels measured by a FET sensor (data shown in FIG. 3J) (r=0.87, P<0.07).

FIG. 16 provides example Temperature sensor responses according to embodiments. The baseline resistance, R₀, at 22° C. (476Ω), where ΔR corresponds to the change in resistance from baseline. The inset shows the corresponding temperature calibration plot.

FIGS. 17A and 17B provide schematics of field-effect transistor (FET) signal acquisition (standard laboratory instrumentation) according to embodiments. FIG. 17A illustrates A commercial source measurement unit (SMU). FIG. 17B illustrates a multi-channel potentiostat with a microcontroller unit (MCU) and two analog-digital converters (ADC) to control two 3-electrode configurations. Transimpedance amplifier (TIA), working electrode (WE), reference electrode (RE), and counter electrode (CE).

FIG. 18 provides a Comparison of field-effect transistor source-drain current (I_{DS}) responses to pH changes from a custom-developed printed circuit board (PCB) vs. a multi-channel potentiostat according to embodiments. The inset shows the corresponding I_{DS} measurements made by the PCB vs. the standard laboratory instrument (i.e., multi-channel potentiostat). All pH measurements were in phosphate-buffered saline.

FIGS. 19A and 19B illustrate example Ex-situ effects of vertical vibration on field-effect transistor sensing responses for different microfluidic channel heights according to embodiments. Channel heights: (FIG. 19A) 170 μm, (FIG. 19B) 340 μm. The vibrational acceleration profiles are presented in the top half of each panel and the sensor responses (pH 7.5) are depicted on the bottom.

FIGS. 20A to 20L illustrate Multi-channel pH data acquisition via a flexible printed circuit board according to embodiments. FIG. 20A provides Real-time recordings of source-drain current (I_{DS}) for a representative field-effect transistor (FET)-based pH sensor with gate voltage (V_{GS}) sweep to track dynamic variations in FET transfer curves in response to a change in pH from 7.4 to pH 7.0 in phosphate-buffered saline (PBS). FIGS. 20B and 20C provide Representative V_{GS} scans (top) and I_{DS} recordings (bottom) at baseline (pH 7.4) and pH 7.0. FIGS. 20D and 20E provide the transfer curves acquired for baseline (pH 7.4) and pH 7.0. FIG. 20F provides The overlaid transfer curves for the baseline (pH 7.4) and pH 7.0 measurements. The inset shows a high resolution view of the overlaid transfer curves demonstrating that each curve is distinguishable. FIG. 20G illustrates Temporal I_{DS} monitoring to track FET responses to changes in pH from baseline (pH 7.4) to pH 7.0 (sensors 1,2 @200 s; sensors 3,4 @580 s) to pH 6.5 (sensors 1,2 @400 s; sensors 3,4 @780 s) in PBS. FIG. 20H provides Mean I_{DS} responses for four FETs showing large device-to-device variations (52% @pH 7.0; 56% @pH 6.5). FIG. 20I provides The same data shown in FIG. 20G normalized to I_{DS} at baseline (pH 7.4). FIG. 20J provides Mean normalized I_{DS} responses for the four FETs continue to show device-to-device variation (36% @pH 7.0; 25% @pH 6.5). FIG. 20K provides The same data in FIG. 20G depicted as calibrated responses with respect to time. FIG. 20L provides Mean calibrated responses for the four FETs show reduced/minimal device-to-device variation (14% @pH 7.0; 3% @pH 6.5).

FIG. 2I illustrates example Characterization of leakage current through a representative gate electrode according to embodiments. Minimal leakage current (I_{GS}) through a Ag/AgCl reference electrode was observed relative to the source-drain current (I_{DS}) in artificial sweat.

FIGS. 22A to 22C provide example Characterization of cortisol in human saliva and sweat samples by liquid chromatography tandem mass spectrometry (LC-MS/MS) according to embodiments. FIG. 22A provides A LC-MS/MS-based cortisol calibration plot. FIG. 22B provides Ion chromatograms of cortisol in a diluted human saliva sample. FIG. 22C provides Ion chromatograms of cortisol in a diluted human sweat sample.

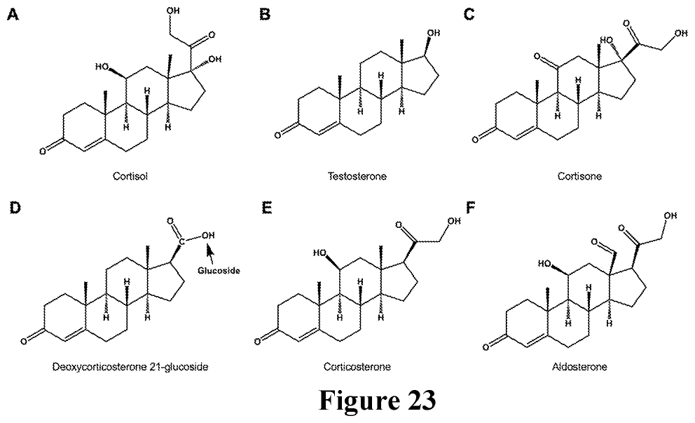

FIG. 23 provides a Summary of chemical structures according to embodiments.

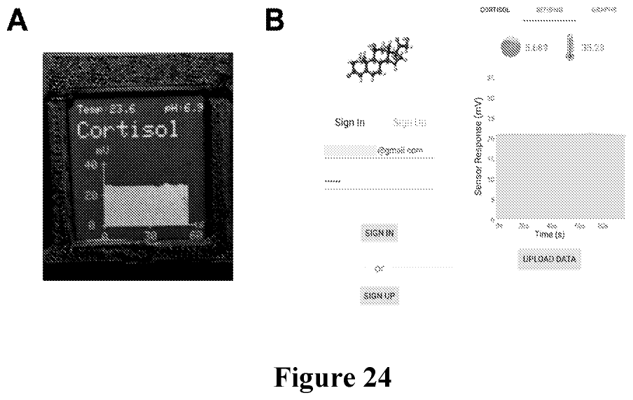

FIGS. 24A and 24B provide example Images for the aptamer-FET biosensing smartwatch and smartphone application according to embodiments. FIG. 24A illustrates a Smartwatch screen. FIG. 24B provides Screenshots of the login and sensing pages of the smartphone Android application.

Example Materials—All chemicals were purchased from Sigma-Aldrich Co. (St. Louis, MO) unless otherwise noted. Prime quality 4″ Si wafers (P/B, thickness 500 μm) were purchased from Silicon Valley Microelectronics, Inc. (Santa Clara, CA). Oligonucleotides (Table S2) were obtained from Integrated DNA Technologies (Coralville, IA). Indium(III) nitrate was purchased from Alfa Aesar (Thermo Fisher Scientific, Waltham, MA) and used as received. The SYLGARD 184 for producing polydimethylsiloxane (PDMS) wells was purchased from Dow Corning Corporation (Midland, MI). Water was deionized before use (18.2 MΩ) via a Milli-Q system (Millipore, Billerica, MA). Anisotropic conductive film (9703, Electrically Conductive Adhesive Transfer Tape, 50 μm) was purchased from 3M (Saint Paul, MN).

TABLE S2
Oligonucleotide sequences.

| | |
|---|---|
| Thiolated cortisol | 5′-/5ThioMC6- |
| aptamer sequence | D/CGACCGGTCTGGGGACCCTGTCTGGGTGTGTGGGTAGTAG |
| | GTCG-3′ |
| Cortisol aptamer | 5′-/56-FAM/CTC TCG GGA CGA CCG GTC TGG GGA CCC TGT |
| sequence with | CTG GGT GTG TGG GTA GTA GGT CGT CCC-3′ |
| fluorescein at the | |
| 5′-end | |
| Quencher strand | 5′- GGT CGT CCC GAG AG/3Dab/-3′ |
| with dabcyl at the | |
| 3′-end | |
| Scrambled cortisol | 5′-/5ThioMC6- |
| aptamer sequence | D/CCACCGCAGTCCGGTCGCTTGCTCGCTGTGTGGGTAGTAG |
| | GTCG-3′ |
| Thiolated serotonin | 5′-/5ThioMC6- |
| aptamer sequence | D/CGACTGGTAGGCAGATAGGGGAAGCTGATTCGATGCGTGG |
| | GTCG-3′ |

Oligonucleotides used in selection process

| | |
|---|---|
| N36 random library | 5′-GGA GGC TCT CGG GAC GAC- (N₃₆)-GTC GTC CCG CCT TTA |
| | GGA TTT ACA G-3′ |
| Biotinylated column | 5′-GTC GTC CCG AGA GCC ATA/3BioTEG/ |
| immobilizing | |
| capture strand | |
| Forward PCR primer | 5′-GGA GGC TCT CGG GAC GAC-3′ |
| Reverse PCR primer | 5′-CTG TAA ATC CTA AAG GCG GGA CGA C-3′ |
| Biotinylated PCR | 5′-/5Biosg/ CTG TAA ATC CTA AAG GCG GGA CGA C-3′ |
| reverse-primer | |

TABLE S3
Cortisol aptamer selection process.

| Rounds | Target | Counter Target | Buffer Condition |
|---|---|---|---|
| 1 | 100 μM, #3 | DOG, 5 μM #16 | PBS + 2 mM MgCl₂ |
| 2 | 50 μM, #3 | DOG, 5 μM #16 | PBS + 2 mM MgCl₂ |
| 3 | 40 μM, #3 | DOG, 5 μM #16 | PBS + 2 mM MgCl₂ |
| 4-5 | 30 μM, #3 | TES, 5 μM #16 | PBS + 2 mM MgCl₂ |
| 6 | 20 μM, #3 | The mixture of DOG/TES, each 5 μM, #16 | PBS + 2 mM MgCl₂ |
| 7 | 10 μM, #3 | No counter target | NxStage pureflow |
| 8 | 5 μM, #3 | Mixture of DOG/TES, each 5 μM, #20 | NxStage pureflow |
| 9 | 3 μM, #3 | Mixture of DOG/TES, each 5 μM, #20 | NxStage pureflow |
| 10 | 3 μM, #3 | Aldosterone, 3 μM, #16 | PBS + 2 mM MgCl₂ |
| 11 | 2 μM, #3 | Mixture of TES/cortisone, each 2 μM, #20 | PBS + 2 mM MgCl₂ |
| 12 | 2 μM, #3 | DOG,2 μM, #20 | PBS + 2 mM MgCl₂ |
| 13 | 1 μM, #3 | TES, 2 μM, #24 | PBS + 2 mM MgCl₂ |
| 14 | 1 μM, #3 | Cortisone, 1 μM, #24 | PBS + 2 mM MgCl₂ |
| 15 | 0.5 μM, #3 | Cortisone, 2 μM, #20 | PBS + 2 mM MgCl₂ |
| 16 | 0.5 μM, #3 | Cortisone, 0.5 μM, #20 | PBS + 2 mM MgCl₂ |
| 17 | 0.4 μM, #3 | No counter target | NxStage pureflow |
| 18-19 | 0.2 μM, #3 | No counter target | PBS + 2 mM MgCl₂ |

In Table S3, the numbers of rounds of selection and counter-selection were determined empirically using the polymerase chain reaction product elution profiles on agarose gels. Sufficient selection was determined to have occurred when significantly brighter PCR bands were observed from target elution steps compared to the last wash and the “counter-target” elutions. The ‘#’ indicate the number of elutions. An artificial dialysis solution (NxStage pureflow) was used where indicated during selection to isolate a cortisol aptamer for kidney dialysis applications, as well as for use in the physiological fluid sensing applications herein. Carried out were new selections because previously reported cortisol aptamers were isolated under high salt conditions (20 mM HEPES, 1 M NaCl, 10 mM MgCl₂, 5 mM KCl, pH 7.5). As such, the previous aptamers showed inadequate target recognition under physiological salt conditions (e.g., in saliva and sweat). The ionic compositions, including NaCl concentrations, of the artificial sweat and artificial saliva solutions used in the experiments herein are shown in Table S4. The target is cortisol. Deoxycorticosterone 21-glucoside, DOG; testosterone, TES; phosphate-buffered saline, PBS. Chemical structures are shown in FIG. 23.

Aptamer selection and characterization—The cortisol aptamer selection was carried out as per previously published methods with modifications to the target concentration and choice of non-targets (Table S3, FIG. 23). The method was based on selection of oligonucleotide sequences that favor solution target association (elution) vs. capture strand binding (retention). Oligonucleotides used in the selection process were (1) an N36 random library: 5′-GGA GGC TCT CGG GAC GAC-(N36)-GTC GTC CCG CCT TTA GGA TTT ACA G-3′, (2) a biotinylated column immobilizing capture strand: 5′-GTC GTC CCG AGA GCC ATA/3BioTEG/, (3) a forward PCR primer: 5′-GGA GGC TCT CGG GAC GAC-3′, (4) a reverse primer: 5′-CTG TAA ATC CTA AAG GCG GGA CGA C-3′, and (5) a biotinylated reverse-primer: /5Biosg/CTG TAA ATC CTA AAG GCG GGA CGA C. See also table S2.

Standard desalted oligonucleotides were used for the library and primers. Modified oligonucleotides (e.g., biotinylation, fluorophore conjugates) were purified by the manufacturer. All oligonucleotides were dissolved in nuclease-free water and stored −20° C. Polymerase chain reaction (PCR) amplifications were run with 1 cycle @95° C. for 2 min, N cycles @[95° C. for 15 s, 60° C. for 30 s, 72° C. for 45 s], and 1 cycle @72° C. for 2 min. In most cases, PCR was carried out over 11±1 cycles. We used commercially available PBS (Corning cat no. 21-040-CV, NaCl 154 mM, Na₂PO₄ 5.6 mM, and KH₂PO₄ 1.058 mM, pH 7.3-7.5) with additional 2 mM MgCl₂ for most selection rounds. Four rounds were carried out with an NxStage pureflow solution (RFP402, NxStage Medical, Lawrence, MA) in place of PBS buffer (table S3). Candidate aptamer sequences identified by selections are shown in Table S4.

TABLE S4
Cloned sequences from the cortisol aptamer selections.

| | | Sequence Alignment | | | |
|---|---|---|---|---|---|
| | | (5′ → 3′) | | | |
| | Sub | Conserved | | Conserved | |
| Grp | group | region | Random region | region | Cnt |
| 1 | | ctctcgggacgac | CGCCAGAAAGAA-----TGAGGATAGGC- | gtcgtccc | 2 |
| | | | TAGGATAGCCTAG | | |
| | | ctctcgggacgac | CGCCAGAAACTTG---- | gtcgtccc | 10 |
| | | | TGAGGATAGGTGTAGCA---CCTAG | | |
| | | ctctcgggacgac | CGCCAGAA- | gtcgtccc | 7 |
| | | | GATCGCATCGAGGATAGTTCACAA----- | | |
| | | | CTAG | | |
| 2 | A | ctctcgggacgac | TACA- | gtcgtccc | 2 |
| | | | TGGGTGTGTGGGTAGGTCTGGGGACCCG | | |
| | | | GTG | | |
| | | ctctcgggacgac | CATGTTGGGTGTGTGGGTAGGTCTGGGGA | gtcgtccc | 1 |
| | | | CCCGGTG | | |
| | B | ctctcgggacgac | CGGTCTGGGGACCCTGTCTGGGTGTGTG | gtcgtccc | 1 |
| | | | GGTAGTAG | | |

In Table S4, twenty-three clones were sequenced. Copy number redundancies and sequence homologies were analyzed (Multali, http://multalin.toulouse.inra.fr/multalin/multalin.html). Lower case letters represent conserved sequence regions. Capital letters indicate the random region. Colored letters show common motifs. Aptamer 2b was used in this study.

The cortisol aptamer sequence (Table S2) was modified with fluorescein at the 5′-end (5′/56-FAM/CTC TCG GGA CGA CCG GTC TGG GGA CCC TGT CTG GGT GTG TGG GTA GTA GGT CGT CCC-3′). The quencher strand was labeled with dabcyl at the 3′-end (5′ GGT CGT CCC GAG AG/3Dab/-3′). The aptamer to quencher ratio (1:5) and assay conditions were as previously described. The cortisol aptamer Kd was determined as described by Hu et al. (J. Hu, C. J. Easley, A simple and rapid approach for measurement of dissociation constants of DNA aptamers against proteins and small molecules via automated microchip electrophoresis. Analyst 136, 3461-3468 (2011)) in PBS with 2 mM MgCl₂ (FIG. 10B).

Embodiments used a thioflavin T (ThT) assay to investigate aptamer specificity (A. Renaud de la Faverie, A. Guedin, A. Bedrat, L. A. Yatsunyk, J.-L. Mergny, Thioflavin T as a fluorescence light-up probe for G4 formation. Nucleic Acids Res. 42, e65 (2014)). Final concentrations in the incubation solutions were aptamer (400 nM), ThT (4 μM), and target or non-targets (0-10 μM) (FIG. 0C). The aptamer was incubated in 95° C. PBS for 5 min (1.6 μM) and cooled to room temperature over 30 min. Aptamer and ThT (16 μM in PBS) were mixed (1:1 ratio) and incubated for 40 min. Targets or non-targets (2× final concentrations in PBS) were added to each oligonucleotide/ThT sample solution. Target/non-target concentrations were tested in triplicate in a final volume of 135 μl. Fluorescence measurements were performed using a Molecular Devices Flexstation II plate reader (Molecular Devices, San Jose, CA) with 425 nm light for excitation and recording emission at 495 nm.

For circular dichroism determination of aptamer secondary structure, aptamer and target concentrations were 1 μM in artificial sweat. Aptamers were thermally treated as described above. Spectra were collected on a JASCO J 715 circular dichroism spectrophotometer (Jasco Products Company, Oklahoma City, OK) at room temperature. Four scans were acquired per sample with 0.5 nm resolution, 1.0 nm bandwidth, a 4 s response time, and a 20 nm/min scan rate. Scans are averages of four instrumental scans and representative of three replicates per condition. Scans in artificial sweat without targets were subtracted as background.

Field-effect transistor fabrication and functionalization—Polyimide films were fabricated using PI-2611 solution (HD MicroSytems, Parlin, NJ). The PI-2611 solution was used as received and was spin-coated onto Si wafers directly at 3000 rpm for 30 s. The film was baked at 150° C. for 90 s, followed by thermal annealing at 350° C. for 30 min in an oven. The polyimide film thickness was ˜7 μm as per the technical information provided by HD MicroSystems for PI-2611 and was confirmed using a profilometer (Dektak 6M profilometer, Bruker, Billerica, MA).

Aqueous solutions (0.1 M) of indium(III) nitrate hydrate (In(NO3)3·xH₂O, 99.999%) were then spin-coated (3000 rpm) for 30 s on flexible polyimide substrates or heavily doped silicon wafers (Silicon Valley Microelectronics, Santa Clara, CA) with 100-nm-thick thermally grown SiO₂ layers. After coating, substrates were pre-baked at 150° C. for 10 min followed by thermal annealing at 350° C. for 4 h. Surface roughness of In₂O₃ was measured using an atomic force microscope (Bruker Dimension FastScan, Billerica, MA) and calculated as the root mean square of peaks and valleys in each measured topographic image (FIG. 7B) using Nanoscope Analysis (Bruker, Billerica, MA). Patterning of In₂O₃ was by photolithography followed by dry etching using a STS advanced oxide etcher (Surface Technology Systems plc, Newport, United Kingdom). Interdigitated source and drain electrodes (1500 μm length, 80 μm width, 10 nm Ti, 30 nm Au) were patterned by photolithography with metal deposition by electron-beam evaporation. After fabrication, the polyimide was delaminated and cut using a razor blade.

Field-effect transistors were functionalized using previously reported protocols. Specifically, (3-aminopropyl)triethoxysilane (APTES) and trimethoxy(propyl)silane (PTMS) (1:9 v/v ratio) were self-assembled on In₂O₃ using vapor-phase deposition. Solutions and devices were placed in a water bath at 40° C. for 1 h followed by baking on a hot plate at 80° C. for 10 min. Devices were then incubated with 1 mM 1 dodecanethiol in ethanol for 1 h to passivate the Au source and drain electrodes. The FETs for pH sensing were used without further modification.

To fabricate aptamer-functionalized FETs, silanized FETs were rinsed with ethanol and immersed in 1 mM 3 maleimidobenzoic acid N hydroxysuccinimide ester (MBS), which was dissolved in a 1:9 (v/v) mixture of dimethyl sulfoxide and PBS (pH=7.4, Gibco, Thermo Fisher Scientific Inc., Waltham, MA) for 30 min. In parallel, thiolated DNA aptamers were prepared by heating at 95° C. for 5 min in nuclease-free water followed by rapid cooling in an ice bath and a return to room temperature. The MBS-modified In₂O₃ surfaces were rinsed with deionized water and immersed in 1 μM thiolated DNA aptamer solutions overnight (>12 h) for aptamer immobilization. The MBS crosslinked amine-terminated silanes with thiolated DNA aptamers. Before measurements, aptamer-FET sensors were rinsed with deionized water and blown dry with N₂ gas.

A scrambled sequence with the same numbers and types of nucleotides as the correct aptamer sequence but with a pseudo-random order was designed to investigate nonspecific aptamer-target recognition on FETs (table S2). The scrambled sequence was selected based on modeling (mfold: http://unafold.rna.albany.edu/?q=mfold) to adopt a significantly different secondary structure compared to the correct sequence.

Field-effect transistor biosensing—For pH sensing, each amine-functionalized FET was immersed in PBS with a Ag/AgCl reference electrode (SDR2, 2 mm diameter, World Precision Instruments, Inc., Sarasota, FL), which acted as the gate electrode (liquid-gate). Sensor measurements were performed using a multi-channel electrochemical workstation (CHI1040C, CH Instrument, Austin, TX). Multiple-channel input was use to obtain transfer curves. To achieve gate-source sweep voltage biasing (VGS), the Ag/AgCl electrode (Channel 1) had an applied linear sweep from 0 V to +0.4 V at 10 mV/s. The counter and reference electrode connectors of Channel 1 were connected to the source electrode of each FET. To achieve a constant drain-source bias voltage (VDS), the drain electrode was connected to the working electrode connector of channel 2 and a constant potential was applied (0.4 V).

Five overlapping transfer curves at each pH were averaged. Calibrated responses (VGS=200 mV) were calculated to minimize device-to-device variations as described in the Results and Supplementary Information. The accuracy of the FET pH sensors was validated by comparing the measured results with corresponding measurements obtained using a standard pH meter (Fisher Scientific AE150).

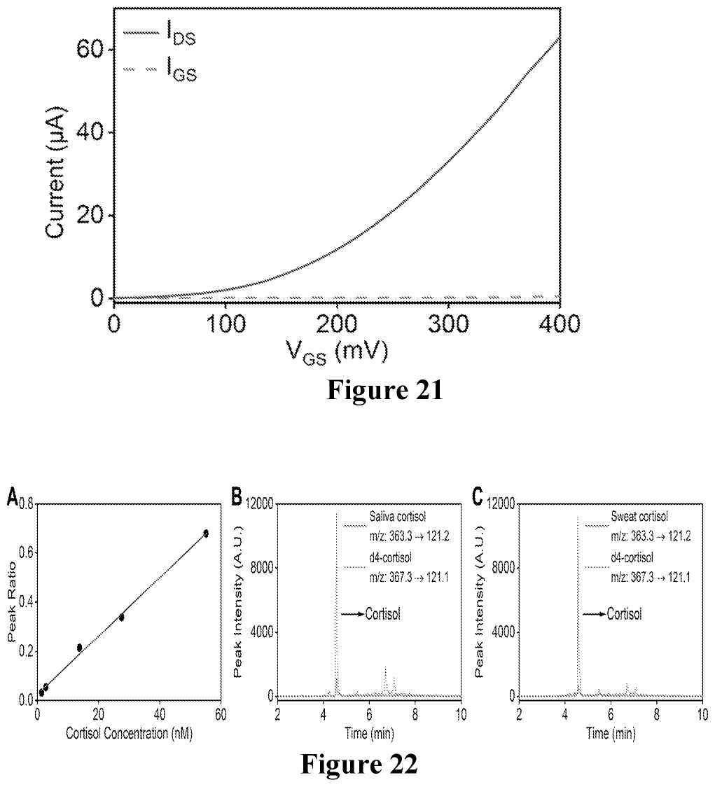

For aptamer-FET measurements, PDMS wells were placed over individual FETs to contain sensing solutions. Artificial saliva (1700-0303, Pickering Laboratories, Inc., Mountain View, CA) or artificial sweat (I2BL-0011, Pickering Laboratories) were used as electrolyte solutions (Table S5). The Ag/AgCl reference electrodes were placed in the sensing solutions above FETs. Sensor measurements were performed using a manual analytical probe station (Signatone, Gilroy, CA) equipped with a Keithley 4200A-SCS semiconductor parameter analyzer (Tektronix, Beaverton, OR). Transfer curves were obtained by sweeping VGS (0 400 mV at 5 mV steps, VDS 10 mV). Five overlapping transfer curves were averaged for each target or non-target concentration. Calibrated responses to minimize device-to-device variations were calculated at VGS=100 mV. Signals acquired by aptamer-FETs (i.e., receptor-target binding) are nonlinear by nature (i.e., described by a Langmuir binding isotherm) and are conventionally represented on a logarithmic scale (M. S. Salahudeen, P. S. Nishtala, An overview of pharmacodynamic modelling, ligand-binding approach and its application in clinical practice. Saudi Pharm. J. 25, 165-175 (2017); E. C. Hulme, M. A. Trevethick, Ligand binding assays at equilibrium: validation and interpretation. Br. J. Pharmacol. 161, 1219-1237 (2010)). Minimal leakage current from the reference electrode was verified (FIG. 21). Any FETs that did not stabilize or showed poor transfer curve characteristics were not used.

TABLE S5
Buffer compositions.
Ionic compositions of artificial sweat (I2BL-0011,
Pickering Laboratories, Inc., Mountain View, CA) and
artificial saliva (1700-0303, Pickering Laboratories).

| | |
|---|---|
| Ion | Concentration (mM) |

Artificial Sweat (pH = 7)

| | |
|---|---|
| Na⁺ | 33.1 |
| Zn²⁺ | 0.0112 |
| Ca²⁺ | 1.00 |
| Fe³⁺ | 0.0046 |
| Mg²⁺ | 0.0553 |
| K⁺ | 6.99 |
| Cl⁻ | 42.1 |
| SO₄²⁻ | 0.069 |

Artificial Saliva (pH = 6.8)

| | |
|---|---|
| Na⁺ | 5.65 |
| Ca²⁺ | 1.02 |
| Mg²⁺ | 0.836 |
| K⁺ | 10.1 |
| Cl⁻ | 19.4 |
| HPO₄²⁻ | 3.33 |
| CO₃²⁻ | 3.84 |

FET bending—A polyimide-FET pH sensor was interfaced with a tape-based thin-film microfluidic structure and connected to a potentiostat with the aid of ACF. The microfluidic structure was first fixed on a flat surface and injected with PBS (pH 7.0 and pH 6.8 for two sets of tests) through the inlet of the microfluidic structure. Transfer curves during flat conditions were recorded. Next, sensors were conformally attached to the surfaces of cylinders with radii of 33 mm, 20 mm, or 15 mm, respectively. Transfer curves for each bending condition were determined. FET sensor gate is driven through an on-chip Ag/AgCl reference electrode, which is fabricated by depositing Ag/AgCl ink (Ercon, Wareham, MA) on the electrodes and heating the modified electrodes at 80° C. for 10 min.

Trier Social Stress Test—Psychological stress was produced by the TSST to induce changes in cortisol levels. Saliva samples for this study were provided from a parent study (N=71) conducted in the Department of Psychology at the University of California, Los Angeles (IRB #14-001311). Participants were at least 18 years old, identified as Black/African American or Hispanic/Latino(a), reported a household income less than or equal to 200% of the federal poverty line, and were fluent in English (for the purposes of delivering the speech task during the lab visit). Exclusion criteria (due to incompatibility with study methods or eating outcomes) included history of an eating disorder, currently adhering to a strict diet, nut or food allergies, current major illness, injury, or mental health diagnosis. Additional exclusion criteria related to incompatibility with salivary cortisol sampling included metabolic or endocrine disease (M. M. Van Eck, N. A. Nicolson, Perceived stress and salivary cortisol in daily life. Ann. Behav. Med. 16, 221-227 (1994)), chronic asthma (Y. S. Shin, J. N. Liu, J.-H. Kim, Y.-H. Nam, G. S. Choi, H.-S. Park, Premier Researchers Aiming New Era in Asthma Allergic Diseases (PRANA) Study Group. The impact of asthma control on salivary cortisol level in adult asthmatics. Allergy Asthma Immunol. Res. 6, 463-466 (2014)), history of substance abuse (S. L. King, K. M. Hegadoren, Stress hormones: How do they measure up? Biol. Res. Nurs. 4, 92-103 (2002)), current use of opiates, steroids (other than inhaled steroids) or anti-psychotic medications (id.), or post-menopausal status (id.).

Participants were scheduled for a laboratory session between the hours of 2:00 PM and 5:00 PM to control for the diurnal pattern of cortisol. The TSST involved two main tasks performed in front of an evaluative audience: (1) public speaking and (2) mental arithmetic. To summarize the protocol briefly, participants were informed about the upcoming tasks and were given 3 min to prepare. They then performed a 5-min speech where the goal was to convince a panel of two evaluators, clad in laboratory coats, that they were the best candidate for a hypothetical job opening. Each speech was videotaped; participants were told their performances would be behaviorally evaluated. Throughout the speech, the evaluators were trained to gaze at participants with neutral faces and at regular intervals, interrupt with sentences such as, “What are your major shortcomings or weaknesses?”

The 5-min mental arithmetic portion required participants to start with the number 2,935 and serially subtract by 7 and then, after 1 min, by 13. Each time a participant made an error, they were instructed to start over at 2,935, and the evaluators were trained to deliver lines such as, “This is just subtraction, try to focus,” throughout the task. The TSST was followed by a 90-min recovery period where the participants watched a neutral documentary.

Saliva (passive drool) was collected at baseline (pre-stress), and 15, 25, and 90 min post-stress. Participants were asked to rinse their mouth with water before saliva collection. At the end of the session, all participants were debriefed and compensated with either course credit or $50. Saliva samples (2 mL) were stored at −20° C. before analysis. Saliva samples were centrifuged at 10,000 rpm for 20 min before cortisol measurements. The samples were analyzed by aptamer-FETs or standard methods (ELISA or LC-MS/MS).

Diurnal saliva/sweat sample collection. Human subject experiments were conducted in compliance with protocols approved by the Institutional Review Board (IRB) at UCLA (IRB #17-000170). All participants gave written informed consent before participation in the study. A pilot study (N=17) was conducted for investigation of cortisol saliva-sweat correlation and validation of cortisol aptamer-FET sensors. Healthy participants were recruited for saliva and sweat collection. Cortisol production undergoes diurnal variation with the highest levels present after waking and the lowest levels present around midnight. Saliva and sweat sample pairs were collected in the morning (˜9:00 AM) and afternoon (˜5:00 PM).

On the day of sample collection, participants were told to report to the laboratory within 1 h of waking and to refrain from food intake at least 1 h prior to sample collection. To collect sweat following a standard protocol, the volar surface of each participant's forearm was cleaned with deionized water and ethanol, followed by sweat gland stimulation using iontophoresis for 5 min. Participants were asked to rinse their mouths with water before saliva collection. Saliva was collected via passive drool after sweat stimulation. Samples were stored at −20° C. until analysis.

Saliva and sweat sample laboratory analyses. Salivary Cortisol ELISA RUO (research use only, SLV2930R, DRG, Inc., Springfield, NJ) or LC-MS/MS were used for the quantitative determination of cortisol in human saliva or sweat. Samples were diluted 1 to 10 fold in sample buffer prior to analysis. For ELISA, assay for cortisol was performed using the manufacturer's protocol. Sensors were tested in artificial saliva (FIG. 12, table S5), which does not contain all species in authentic saliva (e.g., urea). Sensors were tested in real saliva samples (FIGS. 3H and 3J), which contains urea. Artificial saliva was acquired from Pickering Laboratories, Inc (Mountain View, CA) and was formulated according to standard methods (Institut fir Normung 53160).

For LC-MS/MS with multiple reaction monitoring (MRM) analyses, protocols for each biomarker were developed similar to previous work (F. Elio, G. Antonelli, A. Benetazzo, M. Prearo, R. Gatti, Human saliva cortisone and cortisol simultaneous analysis using reverse phase HPLC technique. Clin. Chim. Acta 405, 60-65 (2009); M. Moriarty, A. Lee, B. O'Connell, A. Kelleher, H. Keeley, A. Furey, Development of an LC-MS/MS method for the analysis of serotonin and related compounds in urine and the identification of a potential biomarker for attention deficit hyperactivity/hyperkinetic disorder. Anal. Bioanal. Chem. 401, 2481-2493 (2011)). Human saliva or sweat samples were centrifuged at 14,000 rpm for 10 min and the supernatants were used for analysis. A solid-phase extraction (SPE) technique was used to extract cortisol or serotonin from standard solutions and human saliva or sweat samples (SPE cartridges: Oasis HLB, Waters Corporation, MA). Deuterated cortisol (cortisol-9, 11, 12, 12-d4) or serotonin (serotonin-d4 hydrochloride) were used as the internal standards for quantification of cortisol and serotonin, respectively.

An Agilent 1200 series high performance liquid chromatograph (Agilent Technologies, Palo Alto, CA) equipped with an HTS PAL autosampler (CTC Analytics, MN) was coupled to an API 4000 triple quadrupole mass spectrometer (Sciex, ON, Canada) for MRM experiments. A Zorbax 300 SB-C18 column (0.5 ID×150 mm length, 5 μm particle size, Agilent Technologies) was used for separation. Solvent A was water with 0.1% formic acid; solvent B was acetonitrile with 0.1% formic acid. For cortisol analysis, the flow rate was 400 μL/min with the following gradient: 10% B (0.0-0.5 min), 10 to 90% B (0.5-5.5 min), 90% B (5.5-8.5 min), 90 to 10% B (8.5-9.0 min), 10% B (9.0-11.0 min). For serotonin analysis, the flow rate was 400 μL/min with the following gradient: from 5 to 20% B (0.0-3.0 min), 20 to 90% B (3.0-5.5 min), 90% B (5.5-8.5 min), 90 to 5% B (8.5-9.0 min), 5% B (9.0-11.0 min). Sample vials were maintained at 4° C. in the autosampler tray. A 20 μL aliquot of each sample was injected onto the column.

The instrument was operated in the MRM mode with the following m/z (mass-to-charge) ratio transitions: 363.3→121.1 for cortisol (FIG. 22), 367.3→121.1 for cortisol-d4, 177.2→160.0 for serotonin (FIG. 13), and 181.2→164.2 for serotonin-d4. Peak area ratios of the analytes (cortisol or serotonin) to their respective internal standards were plotted as a function of analyte concentration to construct calibration curves. Analyte concentrations in human saliva or sweat samples were determined based on peak area ratios relative to internal standards and calibration curves. For measurements with each aptamer-FET, the baseline current (artificial saliva or sweat) was collected and then a sample of diluted human sweat or saliva was added so that the final cortisol concentration in the PDMS well was theoretically ˜10 pM (assuming ˜10 nM cortisol in each sample) and sensor responses were collected.

Wireless wearable system design—A dedicated analog, front-end unit was designed and incorporated onto the FPCB to acquire FET transfer curves. Briefly, programmed by the microcontroller unit (MCU) and with the aid of a digital-to-analog converter (DAC), the gate voltage (VG) was periodically swept over the desired range with optionally adjustable biased source and drain voltage levels (VS, VD). The resulting FET IDS was converted to voltage using a transimpedance amplifier with a programmable feedback resistance, effectively implementing a variable gain amplifier (VGA). Similar VGA and voltage biasing configurations were adopted to acquire temperature sensor responses manifested as changes to measured resistance. The output for each of the sensing channels was converted to the digital domain and relayed to the MCU using a high-resolution analog-to-digital converter (ADC) with multiplexer (MUX) front.

In one example design, the DAC (DAC8552, Texas Instruments) was connected to the gate of each FET sensor to perform VGS sweeps (0 400 mV, 10 mV steps @200 ms intervals). The source and drain electrodes of each FET were biased (400 mV) with a potentiostat chip (LMP91000, Texas Instruments, Dallas, TX). The current response (IDS) between the working electrode pin of the potentiostat chip was amplified and converted to voltage by the built-in transimpedance amplifier (programmable TIA, gain: 2.75 kΩ). The analog voltage signal output was converted to the digital domain by a multi-channel 24-bit ADC (ADS1256, Texas Instruments) chip at a sampling rate of 200 Hz. A microcontroller chip (Atmega328, Microchip Technology, Chandler, AZ) was utilized to control the output voltage of the DAC and to collect the readout signal from the ADC by serial peripheral interface communication, where each datapoint was averaged over ten readings.

This circuit board communicated wirelessly and bilaterally with a mobile application user interface on a cell phone via an on-board bluetooth module (AMB2621, Wurth Elektronik, KG, Germany). The acquired and processed sensor outputs were displayed and plotted on a 1.44″ color LCD display (SF-TS144C-9082A-N, Shenzhen SAEF Technology, Shenzhen, China). The entire system was powered by a 110 mAh Li-ion battery (PRT-13853, SparkFun Electronics, Boulder, CO). A smartwatch case was used to hold the sensor array, microfluidic structure, and electronic modules, as well as the battery. The integrated smartwatch was adhered to the wrist with double-sided tape.

Flexible printed circuit board validation—A cortisol aptamer-FET sensor was immersed in a PBS solution and connected to the FPCB. The FET source and drain electrodes were biased at 400 mV. The gate voltage was swept following a staircase waveform from 0 400 mV (10 mV step increments @200 ms). For each step, ten readings were sampled and averaged to obtain the IDS corresponding to each applied VGS. The IDS values were utilized to construct the transfer curves pertaining to each VGS sweep. A solid-state FET (ALD110800, Advanced Linear Devices, Inc., Sunnyvale, CA) was characterized by the FPCB module, potentiostat, and SMU sequentially to verify the FPCB signal acquisition functionality.

Multiplexed measurements with a custom-developed circuit board—For multiplexed pH measurements, two devices (each containing two FET pH sensors) were utilized. Commercial Ag/AgCl reference electrodes were utilized to drive the gates. Each device was immersed in its own beaker with a PBS solution. The four pH sensors were connected to the multichannel on-board SMU for biasing and data recording. Hydrochloric acid was spiked twice in both beakers. Transfer curves for all sensors under different pH conditions were recorded in real-time. The pH values in both beakers were also recorded by a standard pH meter simultaneously. For ex-situ multiplexed measurements with the board, a PDMS well was placed on a polyimide-based FET sensor array, which contained one cortisol sensor, one control sensor (with the scrambled cortisol aptamer), one FET pH sensor, and a temperature sensor. On-chip Ag/AgCl electrode was utilized to drive the gate and fabricated as mentioned above. The custom FPCB was connected to the sensor array to provide biasing. Cortisol solutions were spiked into the PDMS well to change the cortisol concentration to 1 pM and 10 pM sequentially.

Characterization of the wireless FPCB module—A polyimide-FET pH sensor was interfaced with a tape-based thin-film microfluidic device (˜170 μm for each layer) and connected to a custom-developed FPCB with the aid of ACF. The FPCB-connected sensor was then fixed onto a vortex mixer (Fisher Scientific, Waltham, MA) together with an accelerometer (on a smartphone). Artificial sweat (pH 7.2) was injected through the inlet of the microfluidic device to fill the entire structure. Vortical vibrations were introduced by the mixer (5 Hz). Sensor signals were acquired and transmitted wirelessly (via bluetooth) and recorded on a cellphone. Next, artificial sweat pH 7.5 was injected into the microfluidic device to replace the previous solution. The same characterization process was then conducted.

Wearable FET sensing system fabrication—Each FET sensor array was adhered onto the electrical contacts located on the back of the smartwatch using ACF. The FET sensor array was embedded within a tape-based thin-film microfluidic device. Microfluidic channels were created by laser cutting 2D patterns on double-sided tape (˜170 μm, 3M Science, MN; VLS2.30; Universal Laser System, AZ). Outlet features were created by laser patterning holes on polyethylene terephthalate (PET; ˜100 μm; MG Chemicals, Surrey, BC, Canada) to facilitate an ejection path for sampled biofluids. The channel width was 200 μm and the sensing chamber dimension was 3 mm×1.5 mm. The microfluidic device/module was then aligned and assembled by attaching the patterned PET layer to the patterned double-sided tape. It typically took 5 15 min for sweat to fill the microfluidic channels after sweat gland iontophoretic simulation on a 1.2 cm² area of skin. We have utilized similar sweat harvesting strategies for biofluid management and biomarker analysis (e.g., pharmaceuticals and metabolites).

The power consumption of the smartwatch was strongly dominated by the LCD, which had a power dissipation of 0.288 W. The LCD as a heat source was isolated from the sensor by the electronic device and the flexible PCB board. The gap between the LCD and sensor was 3.3 mm. This gap protected the sensor from temperature disturbances. The temperature change on the sensor surface after 10 min of continuous smartwatch operation increased 0.9° C. (from 23.4° C. to 24.3° C.), which should not impact aptamer-FET sensing. We integrated a temperature sensor next to the aptamer-FET array. In future studies, we can investigate the effect of small temperature changes on aptamer-FET responses, and the integrated temperature sensor can be used for correction if there is any response of aptamer-FETs to temperature variation.

Prior to on-body sweat multiplexed measurements, the assembled device was attached to the wrist skin of a healthy subject via double-sided tape and FET sensor baselines were recorded in artificial sweat for self-calibration. To induce sweat iontophoretically, the target stimulation area of the skin was first cleaned with DI water and ethanol, followed by 5 min of iontophoretic sweat gland stimulation (with pilocarpine-loaded hydrogels, Pilogel) using a Macroduct Sweat Collection System (ELITech Group, Puteaux, France). Measurements were conducted at 9:00 AM (1 h after awakening) and 9:30 PM to capture peak and nadir cortisol levels, respectively. The subject refrained from food intake for at least 1 h before each test to avoid confounding effects on body cortisol production. The responses from control sites were subtracted from responses at cortisol sensing sites (R. Zhang, Y. Jia, A Disposable Printed Liquid Gate Graphene Field Effect Transistor for a Salivary Cortisol Test. ACS Sens. 6, 3024-3031 (2021); M. Ku, J. Kim, J.-E. Won, W. Kang, Y.-G. Park, J. Park, J.-H. Lee, J. Cheon, H. H. Lee, J.-U. Park, Smart, soft contact lens for wireless immunosensing of cortisol. Sci. Adv. 6, eabb2891 (2020); N. K. Singh, S. Chung, M. Sveiven, D. A. Hall, Cortisol Detection in Undiluted Human Serum Using a Sensitive Electrochemical Structure-Switching Aptamer over an Antifouling Nanocomposite Layer. ACS Omega, (2021)).

To communicate wirelessly with the FPCB module, an illustrative Android smartphone application was developed (FIG. 24). The application provided a graphical user interface to execute a range of functionalities, including setting the desired operational modes, as well as data display and storage. The Android application was designed to establish communication with the wearable module upon startup. In our implementation, the user input was read with the aid of touchscreen-activated buttons and relayed to the FPCB through the communication of predefined integer values (each value mapped to the desired operation) using Bluetooth. The corresponding commands were received and executed at the microcontroller level. Once communication was established, the user could observe the real-time status of the cortisol, temperature, and pH responses. The real-time and filtered sensing results were then recorded and timestamped in a separate text file on the phone. After the sensing period, the data were uploaded and stored automatically in a Google Cloud Storage bucket.

Statistics—Statistical analyses were carried out in OriginPro (2021, Northampton, MA). Correlations for FET pH sensing vs. pH meter determinations in FIG. 2H, saliva vs. sweat cortisol level correlation in FIG. 3I, and correlations of cortisol levels by aptamer-FETs vs. standard laboratory assays (FIG. 15) were analyzed by Pearson correlations. Data for FIG. 3E were analyzed by one-way ANOVA followed by post hoc Dunnett's multiple comparisons.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for the sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general, such a construction is intended in the sense as one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

What is claimed is:

1. A method of aptamer-FET sensing comprising:
preparing a stem-loop aptamer that contains oligonucleotide sequences with consecutive bases identical at least 80% to GGTCTG or 80% to TGTCTG; and
configuring the stem-loop aptamer to bind to cortisol with a dissociation constant between about 1×10⁻⁸ M to about 1×10⁻⁴ M,
wherein the stem-loop aptamer has at least four times higher binding affinity for cortisol compared to other steroid molecules present in retrievable biofluids.

2. A device comprising:
a flexible or stiff field-effect transistor (FET) biosensor array including:
a cortisol aptamer or an aptamer for another biomarker coupled to a thin-film In₂O₃ FET.

3. The device of claim 2 wherein cortisol levels or other biomarker levels are determined via molecular recognition by the aptamer wherein binding is transduced to electrical signals on the FET.

4. A smartwatch or other wearable device based on the FET biosensor array of claim 2.

5. The smartwatch or other wearable device of claim 4, including a custom, multi-channel, self-referencing, autonomous source measurement unit or other measurement unit enabling seamless, real-time, continuous cortisol or other biomarker measurements in biofluids including sweat, saliva, interstitial fluid, tears, urine, or blood.

6. A device for wearable sensing applications, comprising:
a substrate embedded in a microfluidic device to form a skin-adherable biofluid sampling, routing, and analysis module; and
a cortisol-aptamer-FET sensor or an aptamer-FET sensor for another biomarker formed on the substrate.

7. The device of claim 6, wherein the substrate comprises a flexible polyimide substrate or a substrate comprising another flexible material.

8. The device of claim 6, wherein the sensor comprises an aptamer-FET array.

9. The device of claim 6, further comprising an on-board multi-channel source measurement unit (SMU).

10. The device of claim 6, wherein the sensor comprises quasi-2D FETs employing In₂O₃ or another inorganic semiconductor or graphene fabricated on hard or soft substrates.

11. A method of fabricating a device for wearable sensing applications, comprising:
forming a layer of thin-film In₂O₃ on polyimide via spin coating or other methods of deposition;
patterning the In₂O₃ layer to form channel regions; and
forming source and drain contacts.

12. The method of claim 11, wherein patterning is performed by photolithography or other chemical patterning methods and reactive ion etching.

13. The method of claim 11, wherein forming source and drain contacts includes patterning interdigitated Au/Ti or other metal electrodes.

14. The method of claim 11, wherein the polyimide comprises a flexible substrate.

15. A method of operating a biosensor comprising:
preparing an aptamer-FET sensing interface;
translating target binding events into measurable surface charge perturbations; and
measuring the perturbations as changes in effective VGS and subsequently IDS.

16. The method of claim 15, wherein the target binding events include exposure to human sweat or other retrievable biofluids.

17. The method of claim 15, wherein the target binding events include exposure to human saliva.

18. The method of claim 15, wherein the target binding events include exposure to cortisol or other biomarkers.
